(19) United States
(12) Patent Application Publication
(10) Pub. No.: US 2022/0275434 A1
(43) Pub. Date: Sep. 1, 2022

(54) SYSTEM AND METHOD FOR AMPLIFYING NUCLEIC ACIDS FROM SINGLE CELLS

(71) Applicant: SAMPLIX APS, Herlev (DK)

(72) Inventors: MARIE JUST MIKKELSEN, BRØNSHØJ (DK); ESBEN BJØRN MADSEN, HOLTE (DK); Peter MOURITZEN, Copenhagen V (DK); Thorarinn BLONDAL, BIRKERØD (DK)

(21) Appl. No.: 17/628,092

(22) PCT Filed: Jul. 31, 2020
(86) PCT No.: PCT/DK2020/050223

§ 371 Date: Jan. 18, 2022

(30) Foreign Application Priority Data
Aug. 2, 2019 (DK) PA 2019 00926

(51) Int. Cl. C12Q 1/6848; C12Q 1/6806; B01L 3/00; B01L 3/02

(52) U.S. Cl. CPC C12Q 1/6848; C12Q 1/6806; B01L 3/502761; B01L 3/0268; B01L 2200/027; B01L 2200/0673; B01L 2300/0883; B01L 2400/0439; B01L 2200/0689; B01L 2200/0647

(57) ABSTRACT

The present invention relates to a system for amplification of polynucleotides from a predefined number of single cells. The system comprise a device (or part) providing the predefined number of single cells, at a previously defined inlet site (or orifice) of a cartridge (microfluidic device), and the cartridge itself. The invention further relates to a method for amplification of polynucleotides from the one or more single cells using the system to provide an emulsion of aqueous droplets wherein the nucleic acid amplification occurs. Furthermore, the present invention relates to a kit comprising a plurality of microfluidic devices and a plurality of fluids configured for use with the system and the method.

The present invention relates to a system for amplification of polynucleotides from a single cell comprising a device that deposit a predefined number of cells, preferably a single cell, at a previously defined inlet site of a microfluidic device that is capable of producing an emulsion of droplets when inserted into device that facilitate the generation of an emulsion of droplets.

The invention also relates to a method for amplification of oligonucleotides from a predefined number of cells, preferably a single cell, comprising using the system comprising a device that deposit a predefined number of cells, at a previously defined inlet site of a microfluidic device and a microfluidic device that is capable of producing an emulsion of droplets.

Furthermore, the present invention relates to a kit comprising a plurality of microfluidic devices and a plurality of fluids configured for use with the microfluidic device for provision of emulsion droplets.

Multicellular organisms are made up of different tissues, each of which consists of one or more cellular cell types. The lineage and development stage of a cell determine how it responds to various stimuli, defines the function of the tissue and ultimately the biology of the organism.

Recent research has shown surprisingly great heterogeneity even among cells isolated from the same, apparently homogeneous, tissue, and have stimulated an intensive research pursuing to understand the function by characterizing the DNA and RNA of a single or a few cells.

When analysing the minimal amounts of polynucleotides in a single or a few cells two issues become of paramount importance. One is to avoid loss of the original cellular polynucleotide material; the other is to minimise the risk of polynucleotide contamination which become exceptionally important to avoid, especially if the analysis imply one or more polynucleotide amplification steps.

Likewise, when the analysis implies polynucleotide amplification, it is essential to minimise the amplification bias resulting in non-uniform coverage of sequences that frequently has been described in bulk-amplification setups (Leamon et al. (2006) Nature Methods 3, 541-43).

It has previously been reported that partitioning of molecules, e.g. molecules from a single cell, into a plurality of smaller partitions, e.g. droplets, that both separate the reactions of each cell, enabling processing and analysis of each cell separately, and in addition minimise amplification bias reported to occur during bulk amplification (EP3.314.012; Nishikawa et al. (2015) PLOS ONE|DOI:10.1371/); Rhee et al (2016); Kintses et al. (2010) Cur. Opin. Chem. Biol. 14, 548-555).

However, all of these imply one or more transfer of the original cellular-nuclear acid molecules from one to another container, thus increasing the risk of losing original cellular polynucleotide material and of contamination.

### SUMMARY OF THE INVENTION

The inventors of the present invention have solved the problem of loss of the original cellular polynucleotide material, the risk of polynucleotide contamination and the well-known amplification bias of bulk amplification, by integrating a devise that produces a stream of single cells with a device that deposit the single cell directly at the entry port of a microfluidic device designed to produce very small droplets comprising the necessary reactants for polynucleotide amplification and the microfluidic device.

Thus according to a first aspect of the present invention, there is provided a system for amplification of polynucleotides from a predefined number of single cells, e.g. one single cell. The system comprise a device (or part) providing the predefined number of single cells, at a previously defined inlet site (or orifice) of a cartridge (microfluidic device), and the cartridge itself.

The cartridge comprises one or more groups of containers, wherein each group of containers comprise a supply container, the primary supply container, defining a supply cavity and comprising a primary orifice (or inlet site), an emulsification unit and a collection container.

Each group of containers further comprise a plurality fluid conduits that provide for fluid communication between the primary orifice, the emulsification unit and the collection container, as well as between the secondary orifice, the emulsification unit and the collection container.

The device depositing one or more cells at a previously defined inlet site may comprise more sub-devices or parts. It may e.g. comprise a part which create a flow of single localized cells, a part that focus and eject the cells one at a time, the microfluidic device and a part, e.g. a sample handler, that are able to position the microfluidic device so that a predetermined number of ejected cells hit the primary orifice (or inlet site) of the microfluidic device. The cells may be suspended in an aqueous buffer and a stream of drops in air may be created in a thin tube (as is the case in a flow cytometer or a FACS). Typically, some drops will contain cells, such as one or more cells, and e.g. a FACS can be adjusted eject from the device, one cell comprising drop at a time. A focused ejection cell system (could be a hydrodynamic focusing device), are normally built into the FACS. Such device can be adjusted to provide a predefined number of single localized volumes, e.g. one or more drops, wherein each volume contains a single cell, and deposit the volume at the inlet site of the microfluidic device.

According to a second aspect of the present invention, there is provided a method for amplification of polynucleotides from a predefined number of cells comprising using the system. The method comprise the steps:
i. provide a sample for cells,
ii. prepare a microfluidic device or cartridge by pipetting a volume of cell lysing buffer into or onto an inlet site of the microfluidic device,
iii. insert cartridge into the device that deposit a predefined number of cells into the cell lysing buffer at the microfluidic device,
iv. apply further reactants and use the microfluidic device to form an emulsion of droplets containing a polynucleotide amplification mix, and
v. incubate the emulsion of droplets to obtain amplified nucleic acid from the predefined number of cells.

According to a third aspect of the present invention, there is provided a kit for carrying out the method of the second aspect, which comprises:
a) one or more microfluidic devices (cartridges), each of which comprise one or more groups of containers,
   wherein each group of containers comprise a supply container, defining a supply cavity and comprising a primary orifice (or inlet site), an emulsification unit and a collection container,
   each group of containers comprise a plurality fluid conduits that provide for fluid communication between the primary orifice, the emulsification unit and the collection container;
b) a vial of a suitable oil and a vial of break solution in an amount sufficient to perform the number of reactions provided for by the one or more microfluidic devices (cartridges).

The present invention relates to different aspects including the devices and methods described above and in the following. Each aspect may yield one or more of the benefits and advantages described in connection with one or more of the other aspects. Each aspect may have one or more embodiments with all or just some of the features corresponding to the embodiments described in connection with one or more of the other aspects and/or disclosed in the appended claims.

Other systems, methods and features of the present invention will be or become apparent to one having ordinary skill in the art upon examining the following drawings and detailed description. It is intended that all such additional systems, methods, and features be included in this description, be within the scope of the present invention and protected by the accompanying claims.

### Definitions

Prior to a discussion of the detailed embodiments of the invention a definition of specific terms related to the main aspects of the invention is provided.

Throughout the present disclosure, the term “droplet” refers to an “emulsion droplets”, such as provided according to the present invention. Typically, the droplets are so-called single emulsion droplets, i.e. water-in-oil droplets, and for most purposes the individual droplets have a volume in the nL-range and below. However, in certain embodiments double-emulsion droplets, i.e. droplets comprising an aqueous inner phase and an oil layer being suspended in an outer aqueous carrier phase, are contemplated.

“fluorocarbon oil”, perfluorocarbons or PFCs, are, organofluorine oils typically with a density higher than water. Example of a useable oils are the Fluorinert™ FC-40, Sigma-Aldrich, St. Louis, Mo., USA; Krytox™, Chemours, Wilmington, Del., USA; and Novec™ Oil, 3M Co., Maplewood, Minn., USA.

Herein, the terms “oil”, “emulsion oil” and “carrier fluid” may be used synonymously in the case of single emulsion droplets. In case of double emulsion droplets the carrier fluid is typically an aqeuous fluid.

“dMDA” refer to the multiple displacement amplification (MDA) technique, Blanco et al (1989) 3. Biol. Chem. 264: 8935-40; Zanoli et al (2013) Biosensors 3, 18-43, performed in droplets.

“PCR” refer to the refer to the Polymerase Chain Reaction technique as described in U.S. Pat. No. 4,683,195.

“FACS” is short for fluorescence-activated cell sorter.

“emulsification section” refer to a part of a microfluidic network that may provide an emulsion of aqueous droplets when at least two different types of reactants, a water miscible and a water un-miscible reactant, are brought to flow through the network.

Throughout the text “cartridge” and “microfluidic device” are used synonymously. It refers to a device which comprises a microfluidic network that may form an emulsion of aqueous droplets when provided with suitable reactants and subjected to conditions which make the reactant flow through the microfluidic network. Typically this device are made of two or more parts made from one or more types of polymers such as PMMA (Poly(methyl methacrylate)), Polycarbonate, Polydimethylsiloxane (PDMS), COC Cyclic Olefin Copolymer (COC) e.g. including also TOPAS, COP Cyclo-olefin polymers (COP) including ZEONOR®, Polystyrene (PS), polyethylene, polypropylene, or negative photoresist SU-8. In addition, the cartridge may contain parts made of materials including glass, silicon, or other materials providing hydrophilic properties. In certain situations, it is preferred to make part of the fluidic network hydrophobic. This may be accomplished by siliconization, silanization, or coating with amorphous fluoropolymers, or alternatively by applying a layer of Aquapel, sol-gel coating, or by deposition of thin films of gaseous coating material.

While the invention is mostly illustrated with a microfluidic device that produce single emulsion droplets the invention may as well be embodied applying a microfluidic device which produced double emulsion droplets.

The term “microfluidic” imply that at least a part of the respective device/unit comprises one or more fluid conduits being in the microscale, such as having at least one dimension, e.g. the width and/or height height, being smaller than 1 mm and/or having a cross-sectional area smaller than 1 mm². Preferable less than 500 μm or with a cross-sectional area smaller than 500 μm², such as less than 200 μm with a cross-sectional area smaller than 200 μm².

A further understanding of the nature and advantages of the present invention may be realized by reference to the remaining portions of the specification and the accompanying drawings. In the following, preferred embodiments of the invention are explained in more detail with reference to the drawings, wherein:

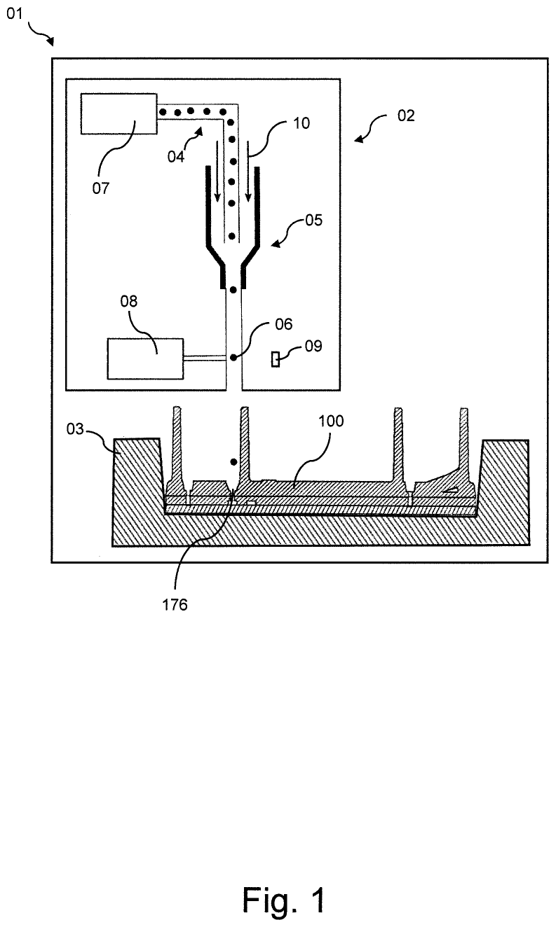

FIG. 1 schematically illustrates a system according to the invention for amplification of polynucleotides from a single cell and which comprise a part that provides one or more single cells into a microfluidic device.

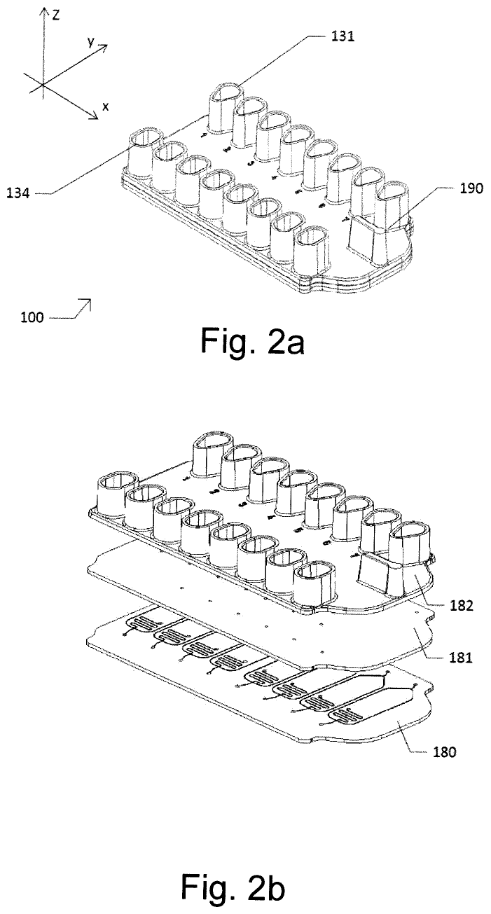

FIG. 2 schematically illustrates a side view of a first embodiment of the microfluidic device subpart of the system. FIG. 2a illustrates a side non-exploded view of the microfluidic device, when it is assembled and FIG. 2b shows an exploded view of FIG. 2a.

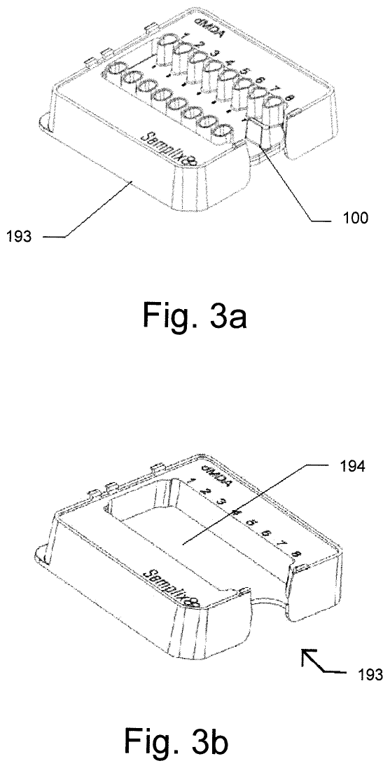

FIG. 3a illustrates an embodiment wherein the microfluidic device is inserted into a holder or housing. FIG. 3b shows the housing without the microfluidic device.

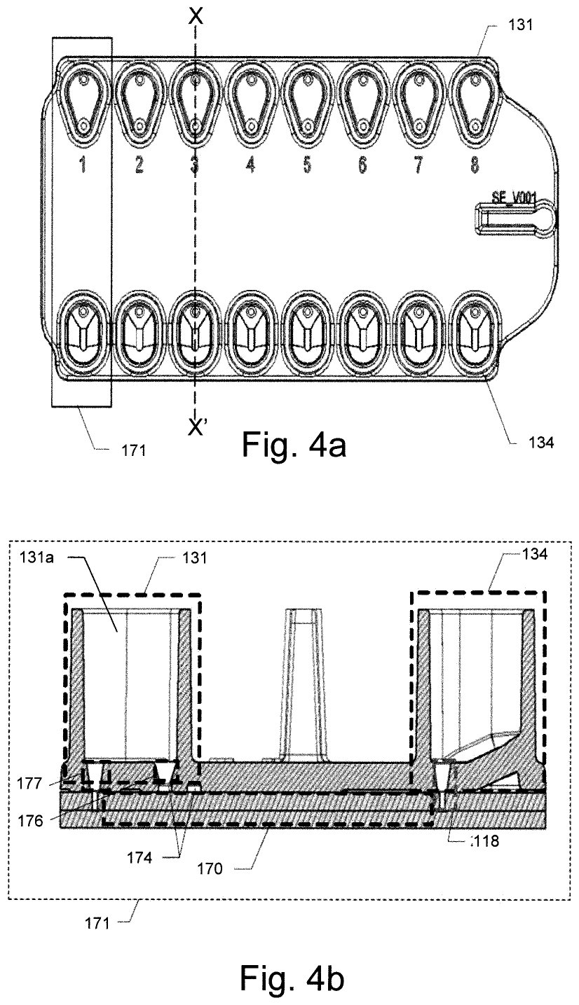

FIG. 4 shows an embodiment of a microfluidic device according to FIG. 2. FIG. 4a shows the top view illustrating individual containers. FIG. 4b shows the cross-sectional X-X′ view of the device illustrated in FIG. 4a.

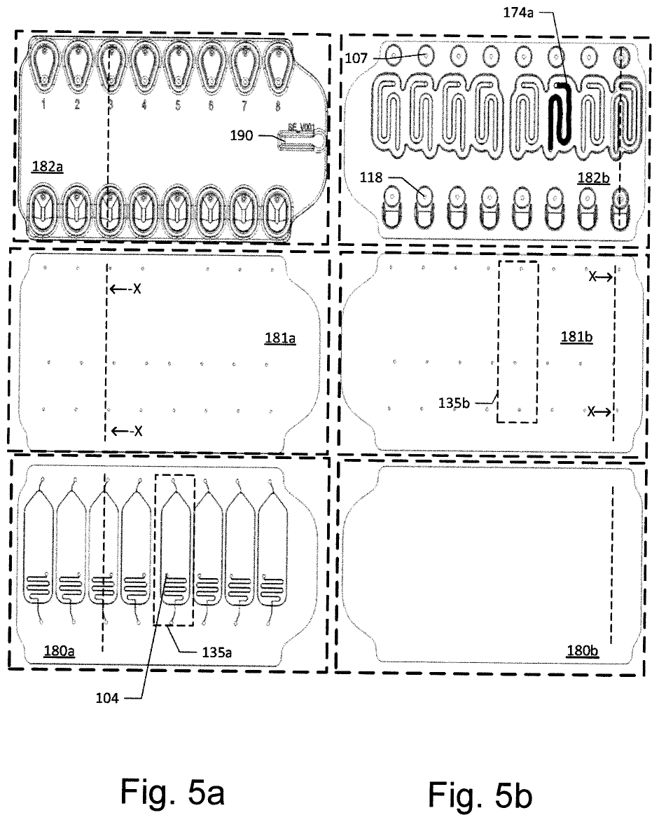

FIG. 5 illustrates the individual parts of the first embodiment of the microfluidic device. FIG. 5a is an exploded view of showing all of the pieces as seen from the top and FIG. 5b shows the exploded view of the pieces as seen from the bottom.

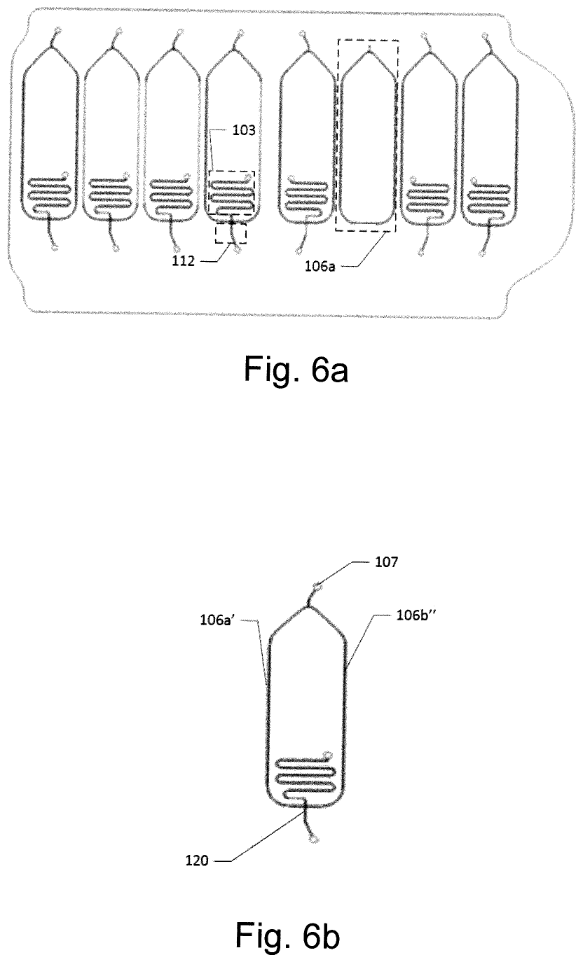

FIG. 6 show an enlargement of the microfluidic network also indicated in FIG. 5. FIG. 6a shows an enlarged drawing of the piece illustrated in FIG. 5a lower panel, and FIG. 6b shows an enlargement of the individual fluid conduit network also illustrated in FIG. 5a.

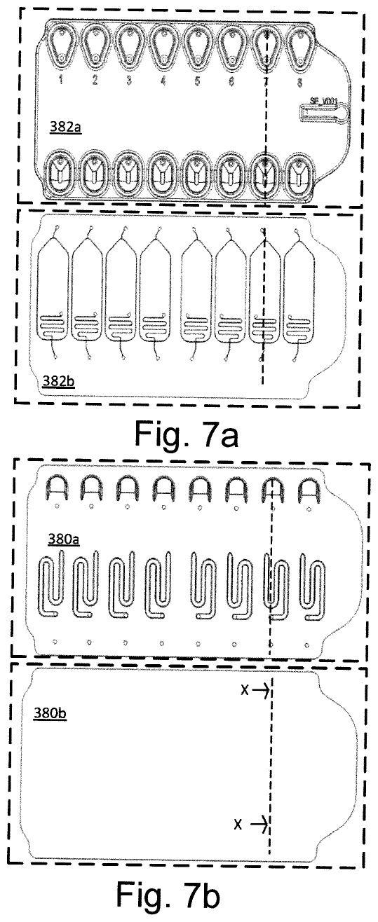

FIG. 7 illustrates the individual parts of a second embodiment of the microfluidic device but made from only two pats. FIG. 7a is an exploded view of showing the upper and the lower piece seen from the top; and FIG. 7b is an exploded view of showing the upper and the lower piece seen from the bottom.

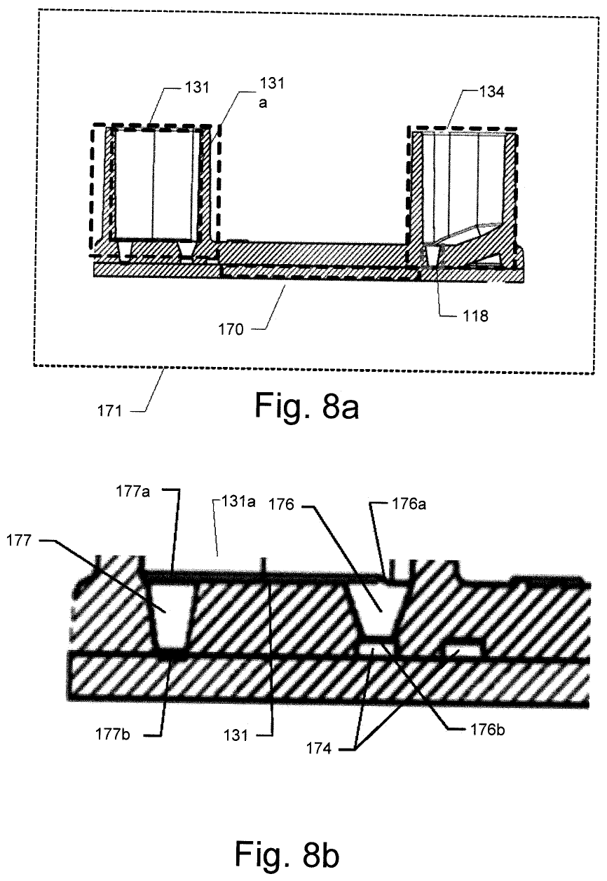

FIG. 8 shows a cross-sectional view of a group of containers according to a second embodiment of the present invention. FIG. 8a shows the cross-sectional view X-X′ of FIG. 7, and FIG. 8b illustrates the enlargement of the primary supply container showing individual parts.

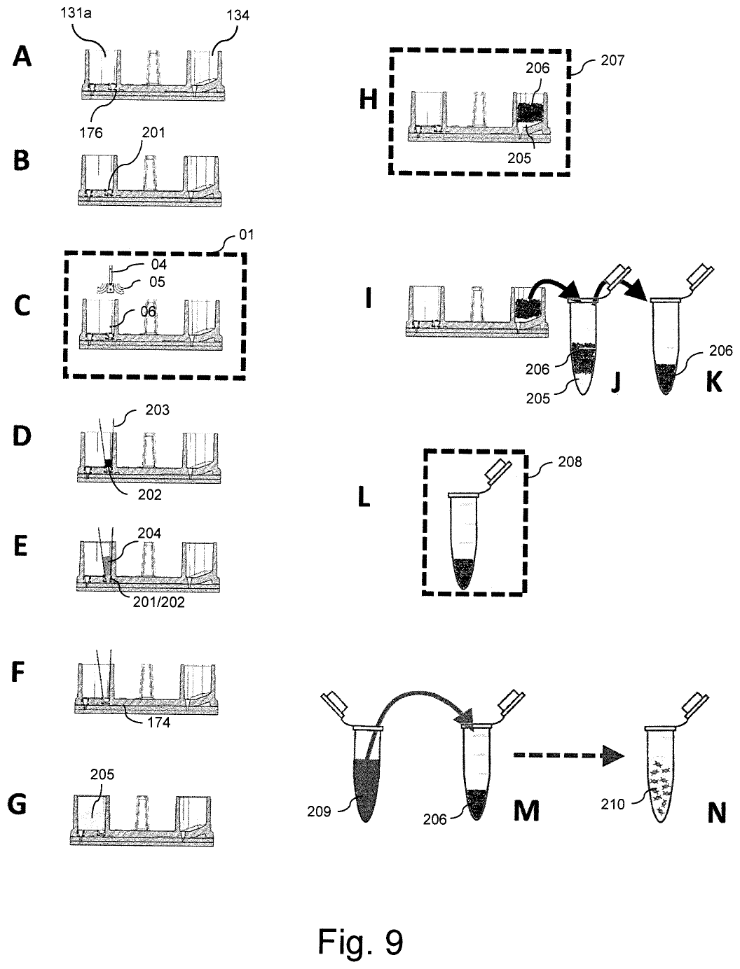

FIG. 9 illustrates the steps in the method.

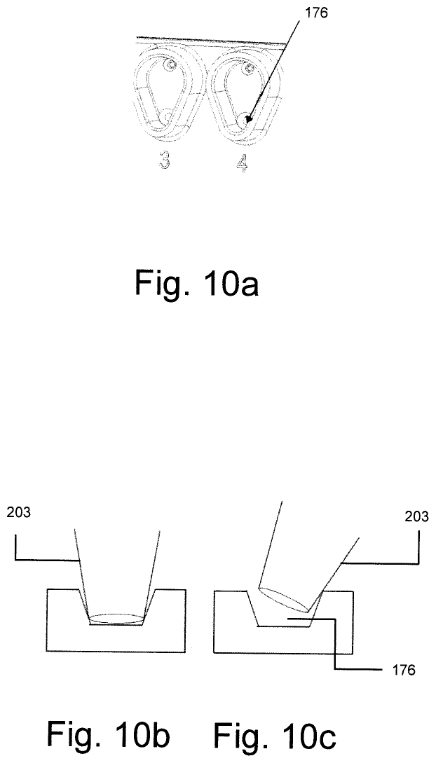

FIG. 10 illustrates the injection of the sample in the inlet well. FIG. 10a shows the position of the inlet well of the primary orifice in certain embodiments as seen from above. FIG. 10b shows the correct insertion of the wide bore pipette tip in the inlet well, and FIG. 10c illustrates an incorrect insertion.

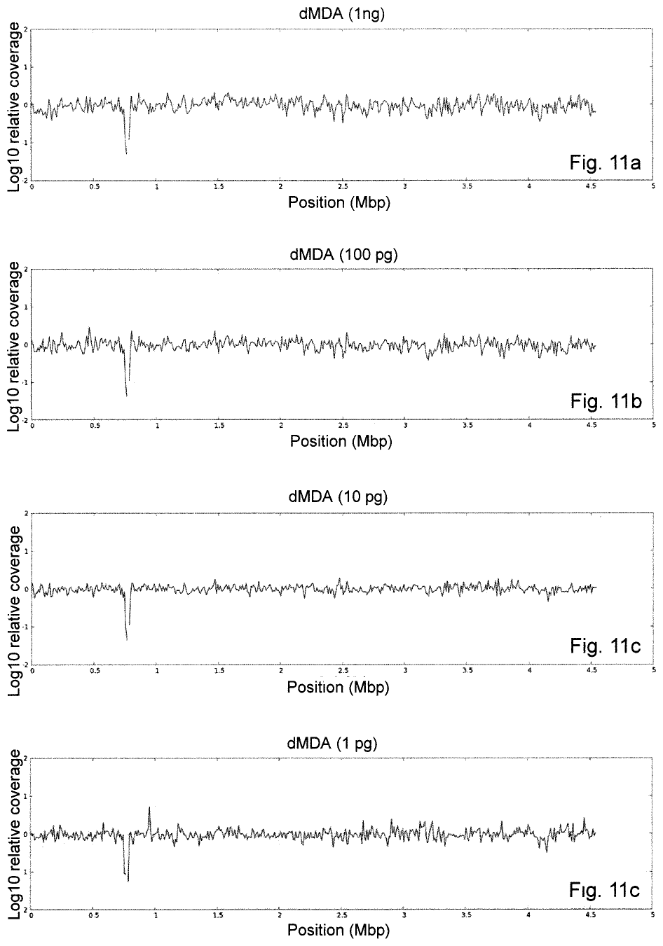

FIG. 11a-d show the result when various amounts of E. coli chromosomal DNA were amplified by the droplet multiple displacement amplification (dMDA) protocol. The y-axis is Log 10 to the relative coverage expressed as cov_{log 10}. The x-axis indicates the position in the E. coli chromosome. FIG. 11a shows the result when 1 ng E. coli DNA is amplified. FIG. 11b shows the result when 100 pg E. coli DNA is amplified. FIG. 11c shows the result when 10 pg E. coli DNA is amplified, and FIG. 11d shows the result when 1 pg E. coli DNA was amplified.

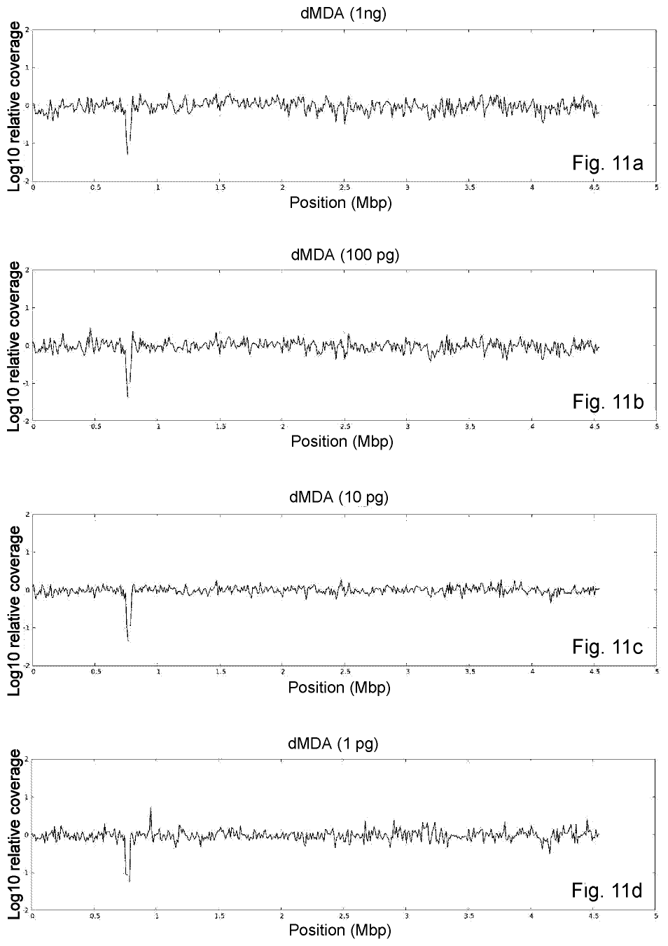

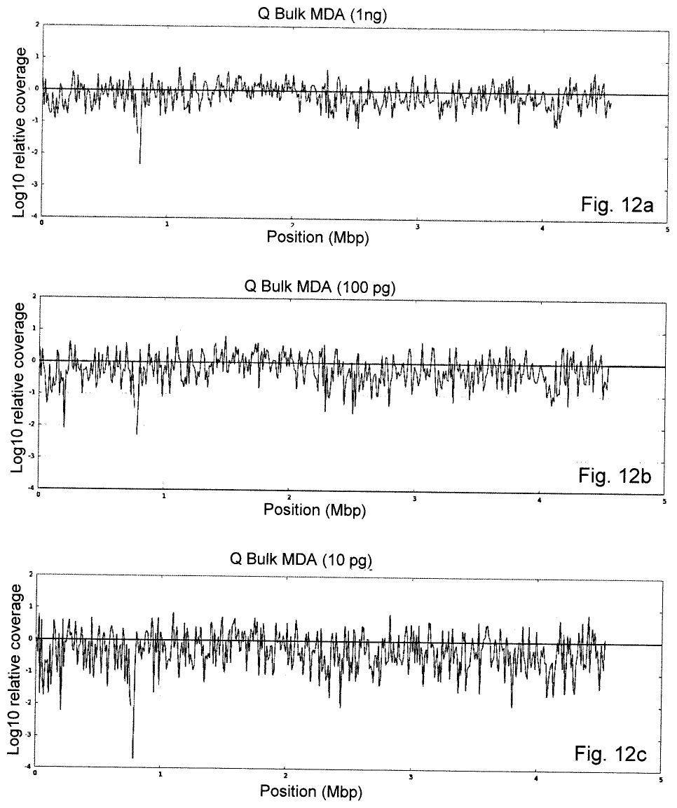

FIG. 12a-c show the result when various amounts of E. coli chromosomal DNA were amplified by the Q bulk multiple displacement amplification (MDA) protocol. The y-axis is Log 10 to the relative coverage expressed as cov_{log 10}. The x-axis indicates the position in the E. coli chromosome. FIG. 12a shows the result when 1 ng E. coli DNA is amplified. FIG. 12b shows the result when 100 pg E. coli DNA is amplified. FIG. 12c shows the result when 10 pg E. coli DNA was amplified. No amplification was observed when amplification of 1 pg E. coli DNA was attempted.

FIG. 13a-c show the result when various amounts of E. coli chromosomal DNA were amplified by the N bulk multiple displacement amplification (MDA) protocol. The y-axis is Log 10 to the relative coverage expressed as cov_{log 10}. The x-axis indicates the position in the E. coli chromosome. FIG. 13a shows the result when 1 ng E. coli DNA is amplified. FIG. 13b shows the result when 100 pg E. coli DNA is amplified. FIG. 13c shows the result when 10 pg E. coli DNA is amplified. No amplification was observed when amplification of 1 pg E. coli DNA was attempted.

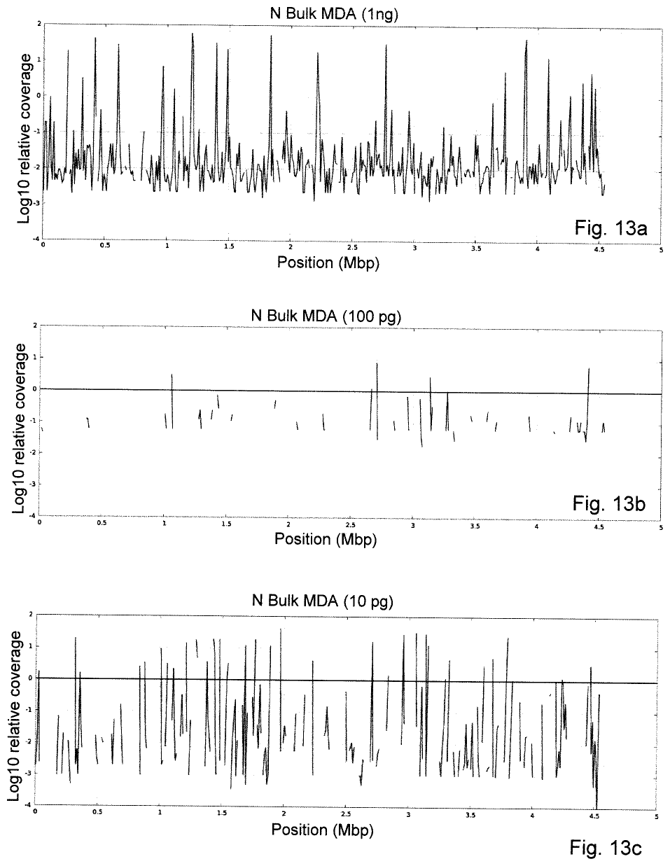

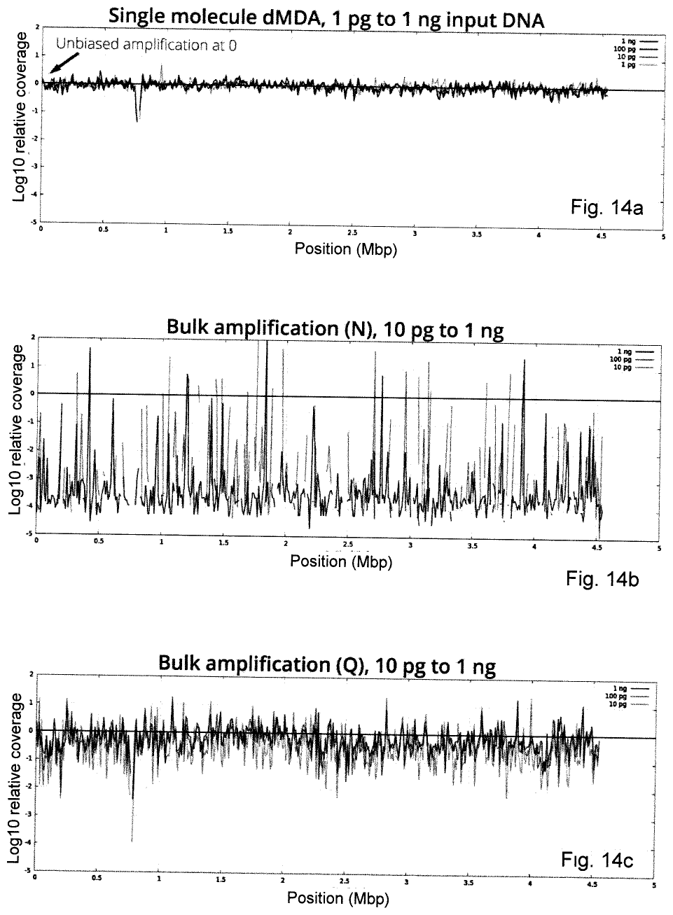

FIG. 14a-c show the result when E. coli chromosomal DNA were amplified by the droplet-MDA, N-bulk MDA and the Q-bulk MDA protocols. The results obtained with 1 pg, 10 pg, 100 pg, and 1 ng E. coli DNA are pooled.

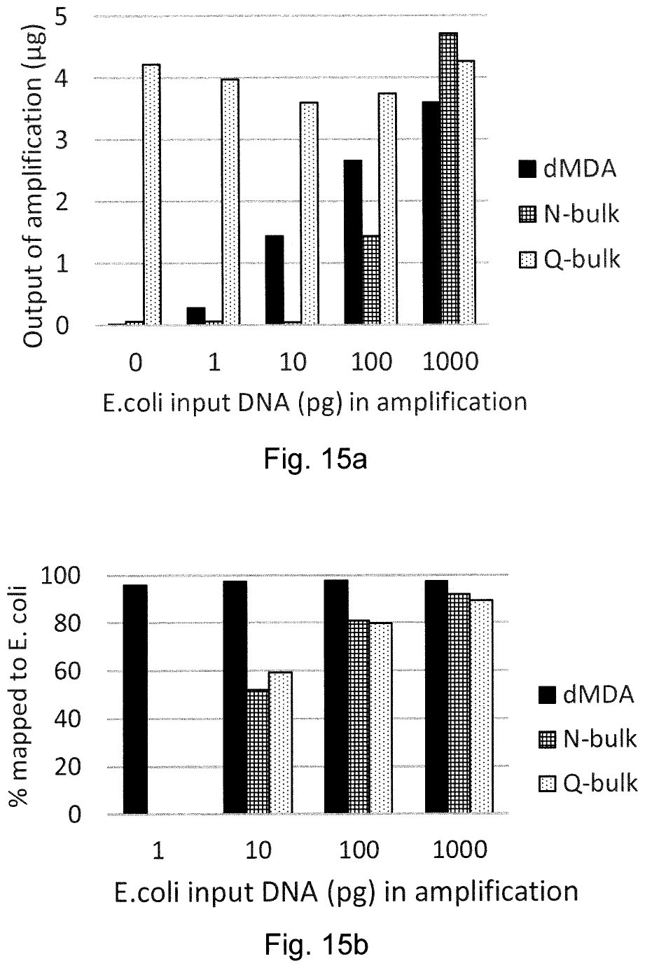

FIG. 15 shows the result when E. coli chromosomal DNA was amplified by the droplet-MDA, N-bulk MDA and the Q-bulk MDA protocols. The results obtained with various amounts of E. coli DNA are shown.

FIG. 15a shows the amount of DNA in microgram obtained by the 3 amplification protocols when starting with 0 pg, 1 pg, 10 pg, 100 pg, or 1 ng E. coli DNA.

FIG. 15b shows the percentage of the amplified DNA in microgram that mapped to the E. coli reference genome obtained by the 3 amplification protocols when starting with 1 pg, 10 pg, 100 pg, or 1 ng E. coli DNA.

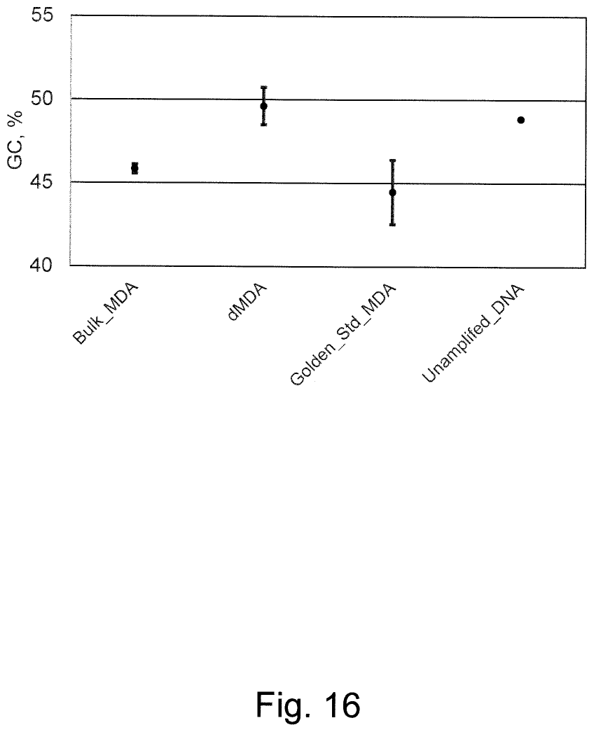

FIG. 16 show the GC % determined by Whole Exome Sequencing (WES) of HT 29 genomic DNA.

“Bulk_MDA” is the GC % og DNA from single Cells subjected to MDA without droplet formation.

“dMDA” is the GC % of MDA-amplified DNA from a single cell according to the present method.

“Golden_Std_MDA” is the GC % of DNA from single cells that were amplified by use of the REPLI-g Single Cell Kit and protocol.

“Unamplified_DNA” is the GC % determined from sequencing of 2 pg unamplified DNA.

Note all experiment were in quatroduplicate except the “unamplified DNA” which comprised only one sample. Dot and error bar indicate mean and 1× standard error.

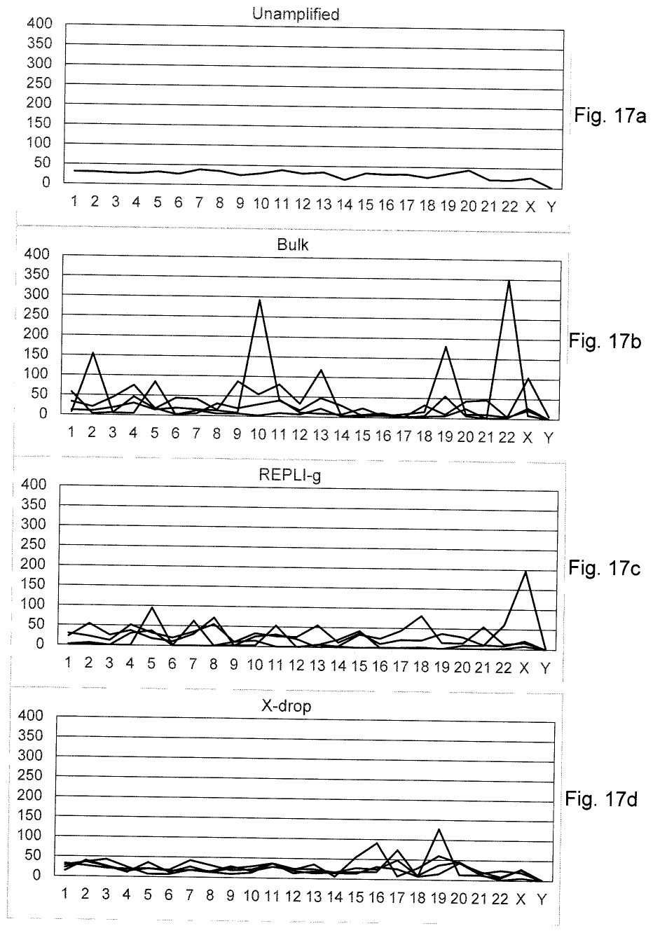

FIG. 17 show the mean coverage depth per chromosome across the human genome. The chromosome designation is indicated at the x-axis.

FIG. 17a: Nonamplified genomic DNA (Unamplified),

FIG. 17b: four separate samples of batch-amplified DNA each of which was from a single Cell amplified by use of Samplix′ dMDA Kit in bulk.

FIG. 17c: four separate batch-amplified DNA's each of which was from a single Cell subjected to MDA. The samples were amplified using the REPLI-g Single Cell DNA kit.

FIG. 17d: four separate samples each of which was from a single Cell amplified by the present Droplet MDA protocol (XDrop-dMDA) i.e. amplified by use of Samplix′ dMDA Kit (Cat. #RE20300) in an emulsion of droplets.

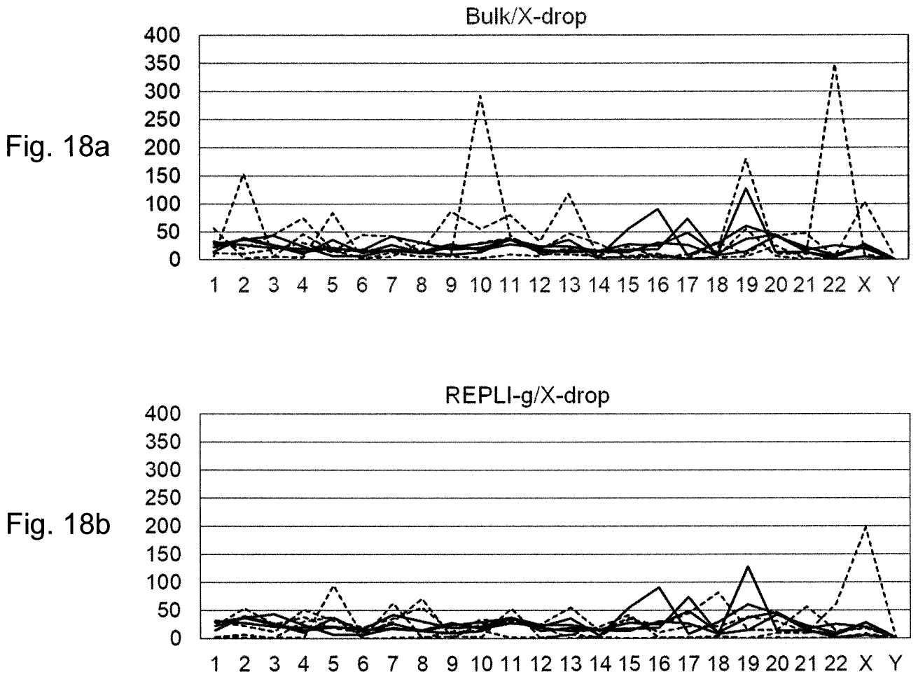

FIG. 18 is a comparison of the present invention, a droplet based MDA procedure, with two different two batch-amplified procedures. In FIG. 18a the data from the Bulk-MDA—broken line, and from Xdrop dMDA solid line, are superimposed. In FIG. 18b the data from REPLI-g—broken line, and Xdrop dMDA—solid line, are superimposed.

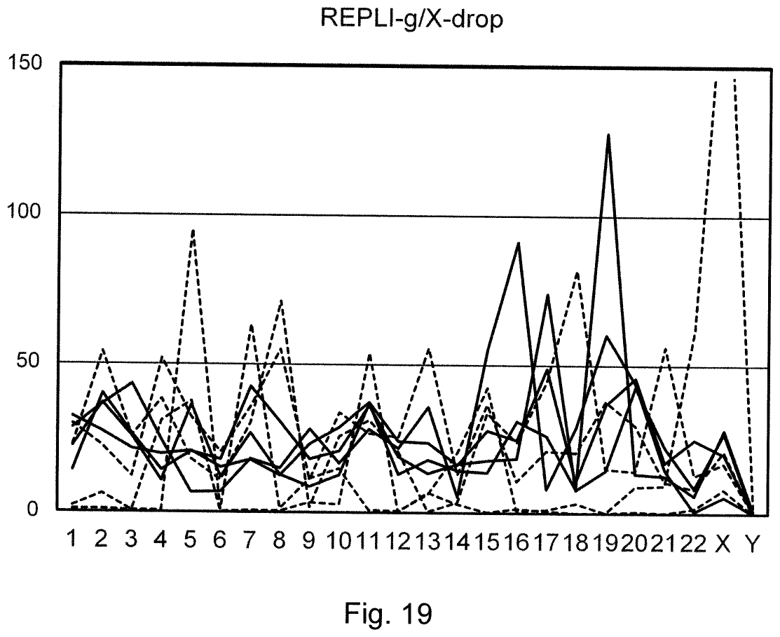

FIG. 19 is a closeup of the data also shown in FIG. 18b. The data from REPLI-g—broken line, and data obtained by the present invention, the Xdrop dMDA—solid line—are superimposed.

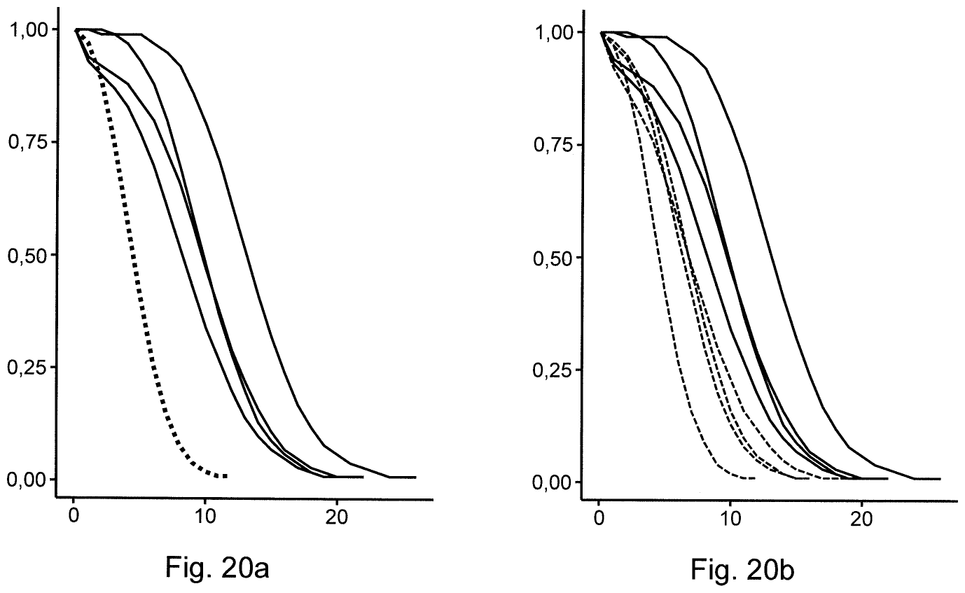

FIG. 20 a and b show the fraction of the exome covered (y-axis) as a function of the depth of coverage.

The dotted line in FIG. 20a illustrates the result obtained from sequencing of 2 μg unamplified HT29 DNA.

Both in FIGS. 20a and b, the 4 unbroken lines illustrate the result from 4 separate samples of MDA-amplified DNA obtained from a single cell according to the present method (Xdrop dMDA).

The 4 broken lines in FIG. 20b illustrate the result from 4 separate samples of MDA-amplified DNA from a single Cell subjected to MDA without prior droplet formation.

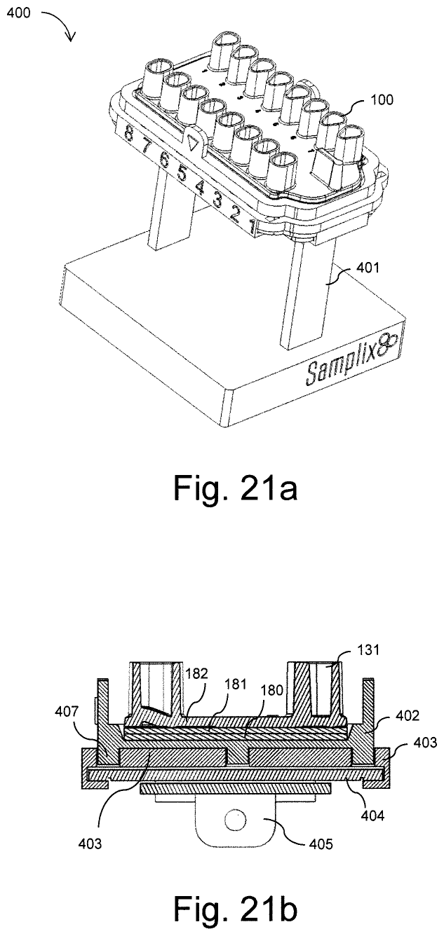

FIG. 21 schematically illustrates one embodiment of the FACS adaptor with a dMDA cartridge inserted. FIG. 21a is a side-view. FIG. 21b is cross-section of the upper part of the FACS adaptor with a dMDA cartridge inserted.

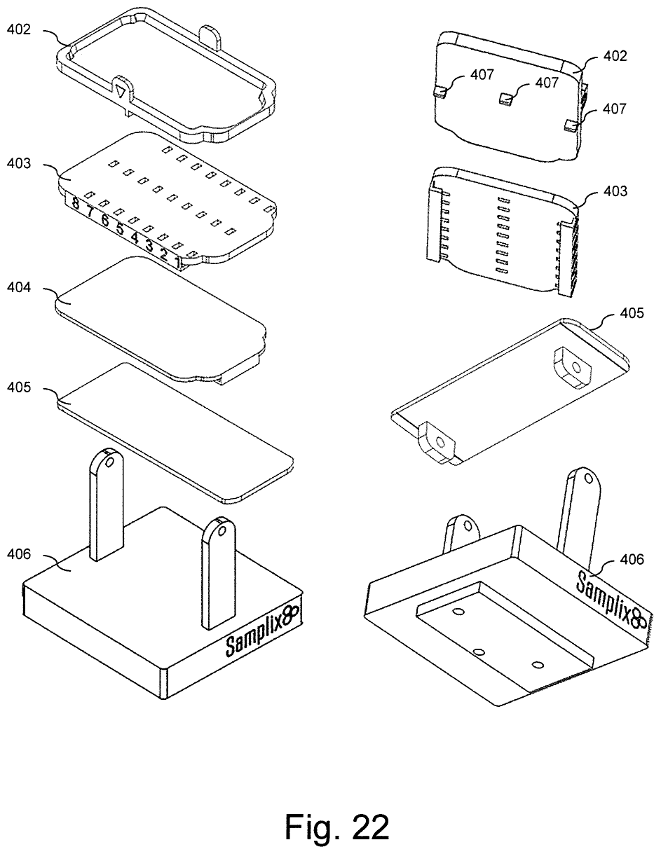

FIG. 22 shows two exploded side views of the FACS adaptor seen from either top (left) or bottom (right).

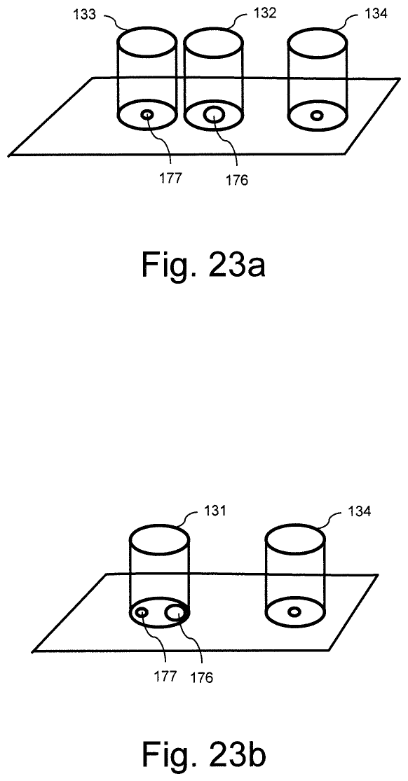

FIG. 23a illustrates a cartridge with separate primary and secondary supply containers. FIG. 23b illustrates a cartridge with the primary and the secondary supply containers are combined into one supply container.

### DETAILED DESCRIPTION

The present invention will now be described more fully hereinafter with reference to the accompanying drawings, in which exemplary embodiments of the invention are shown. The invention may, however, be embodied in different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout. Like elements will, thus, not be described in detail with respect to the description of each figure.

One preferred embodiment of the system [01] for amplification of polynucleotides from predefined number of single cells is shown in FIG. 1. It comprises a device (or part) [02] providing a flow of single localized cells which ejects from the device, one cell at a time, and deposit them at a previously defined inlet site (or orifice) of a cartridge (or microfluidic device) [100], and the cartridge. This embodiment may further comprise a sample handler [03] which is designed to hold the microfluidic device [100] and to move the microfluidic device in response to a signal from the detector [09] to obtain that a predefined number of cells (typically one) are deposited in the individual primary orifices (or inlet sites) [176] of the microfluidic device.

The device (or part) providing a flow of single localized cells, and the device (or part) that provides one or more localized volumes, each volume comprising a single cell at a previously defined inlet of a cartridge (microfluidic device), these two parts may be one or more separate units assembled into a functional unit or one integrated device.

Examples of devices which may be adapted to provide one or more single localized volumes, each comprising a single cell, at a previously defined inlet of a cartridge (microfluidic device) may be selected from the group of devices consisting of a flow cytometer, a fluorescence-activated cell sorting device (FACS), single cell “inkjet” devices (e.g. the x.sight instruments of Cytena GmbH, Freiburg, Germany), acoustic bioprinters, Single Cell Dispensers (e.g. the Namo or Hana Single Cell Dispensers of Namocell Inc., Mountain View, Calif., USA) and micromanipulator-devices (e.g. the CytoPicker™ device, Cytotracks, Lyngby, Denmark).

The output from some of these devices may require an additional focusing of the ejected cell-comprising volumes in order to obtain the precision needed for the present invention. Examples of such additional focusing ejection cell systems are hydrodynamic focusing devices, piezo-driven droplet generating devices (e.g. as described in EP 2 577 254 B1), optical tweezer devices, acoustic tweezer devices (e.g. as described in US 2005/0130257 Al) and photoacoustic tweezer devices.

Some FACS-devices may, in addition to be able to provide a localized volume with a single cell at the inlet of a microfluidic device, also comprise a high precision sample loading device.

Typically, such sample handlers fit the 96- or 384-well plate format. FIG. 3 illustrates one embodiment of a holder [193] configured to hold the microfluidic device [100] in the sample handler of the system. To ensure that temperature-dependent reactions do not occur while the microfluidic device is inserted into the sample handler of the system the holder may be made to provide a thermal connection [194] between the thermal structure and a bottom part of the microfluidic device [100]. Thereby the bottom of the microfluidic device can be cooled down and reactions inhibited.

FIGS. 2 to 8 schematically illustrate various views of some embodiments of a microfluidic device (or cartridge) [100] according to the present invention. The microfluidic device (or cartridge) [100] comprise one or more groups of containers [171], wherein each group of containers comprise a supply container [131], the primary supply container, defining a supply cavity [131a] and comprising a primary orifice (or inlet site) [176], an emulsification unit [170] and a collection container [134].

Each group of containers comprise a plurality of fluid conduits that provide for fluid communication between the primary orifice [176], the emulsification unit [170] and the collection container [134].

The microfluidic device (or cartridge) also comprises a secondary supply container defining a secondary supply cavity. The secondary supply container comprises a secondary orifice [177] extending from the secondary supply cavity and which is in fluid communication with the emulsification unit [170].

In a preferred embodiment the primary and the secondary supply container is integrated to form a combined supply container [131] defining one supply cavity. Se FIG. 23 a and b. When applied to the present procedure it is advantageous that first the sample, then the oil is sequentially added to the same supply well, because when subjected to pressure in the subsequent step, the oil act as “liquid plunger” helping to force the sample into the cartridge.

FIG. 2a shows one preferred embodiment of the microfluidic device (or cartridge) for production of single emulsion droplets having a supply [131] and a collection container [134]. In this embodiment the cartridge (or microfluidic device) is designed with 8 supply and 8 collection containers, forming 8 groups of containers in order to provide 8 individual emulsifications. In addition, this embodiment comprises a handle [190] to facilitate a convenient handling of the device.

FIG. 2b shows an exploded view of the preferred embodiment of the microfluidic device. The figure illustrate that the device may be assembled from 3 individual parts, an upper piece [182], an intermediate piece [181], and a lower piece [180]. Part of the microfluidics that forms the emulsification-part of the device is also indicated at the lower piece.

The 3 individual parts that comprise the functional microfluidic device is further illustrated in FIG. 5.

FIG. 4 further illustrates a top view of the embodiment of the microfluidic device according to FIG. 2, being designed to perform 8 individual emulsions.

FIG. 4a is a top view showing the supply containers [131], the collection containers [134], and a group of containers [171].

FIG. 4b shows the cross-section X-X′ of a group of containers [271] indicated in FIG. 4a.

Some of the functional different parts of the device is indicated by boxes drawn with a broken line.

The supply [131] container defines a supply cavity [131a], it comprise a primary orifice (or inlet site) [176] and a secondary orifice (secondary inlet site) [177].

Referring to FIG. 4b, the primary orifice [176] of the cartridge is configured for accommodating a distal end zone of a pipette tip and is configured for forming a seal with a pipette tip when the distal end zone of the pipette tip is inserted into and pressed against the primary orifice [176]. This may be accomplished by designing the orifice as a truncated cone with an upper opening slightly larger than the opening of a relevant wide-bore pipette tip. Thus, the primary orifice [176] is conical and tapering in a direction away from the secondary supply cavity.

The supply container is in fluid communication with an intermediate chamber [174] of the same group of containers via the primary orifice [176].

Each group of containers comprise an emulsification unit [170] which is in fluid communication both with the intermediate chamber [174] and with the secondary orifice [177]. Also, the collection container [134] is in fluid communication with the emulsification unit [170].

FIGS. 5 and 6, provide further details of an preferred embodiment of the microfluidic device (or cartridge).

FIG. 5 shows an exploded view of all of the pieces of the embodiment of the microfluidic device (or cartridge) also illustrated in FIG. 2.

FIG. 5a show the pieces as seen from the top, FIG. 5b show the pieces as seen from the bottom.

The top two panels of FIGS. 5a and 5b show the upper piece seen from the top [182a] and from the bottom [182b], respectively. Also shown is one embodiment of the intermediate chamber [174a], a secondary supply inlet [107], and a collection outlet or collection orifice [118] as seen from the bottom.

The fluid conduit network of the microfluidic device (or cartridge) is illustrated in FIG. 5. In this embodiment the microfluidic conduit network is formed when the top [182a and 182b], middle [181a and 181b] and the bottom [180a and 180b] parts are assembled. However, the microfluidic conduit network may be formed otherwise in other embodiments.

The broken line box [135b] in FIG. 5b indicate the location of the emulsifying microfluidic network when the middle part [181b] is seen from below.

The broken line box [135a] in FIG. 5a indicate the location of the emulsifying microfluidic network when the bottom piece is viewed from above [180a]. Reference no. [104] indicate the primary supply inlet to the microfluidic conduit network from the intermediate chamber.

Further details of the microfluidic network in the preferred embodiment may become aware from FIG. 6.

FIG. 6a shows an enlarged drawing of the lover piece also illustrated in FIG. 5a.

FIG. 6b shows an enlargement of one individual fluid conduit network also illustrated in FIG. 5a, [135a].

FIG. 6a shows an enlarged drawing of the lower piece seen from the top also illustrated in FIG. 5a. The three broken-line boxes indicates the primary supply conduit [103], which creates fluid communication between the intermediate chamber and the first fluid junction; the transfer conduit [112], which provides fluid communication between the first fluid junction and the collection orifice [118]; and the first secondary supply conduit [106a], which is responsible for fluid communication between the secondary supply orifice [177] and the first fluid junction [120].

FIG. 6b is an enlargement of an individual fluid conduit network also illustrated in FIGS. 5 and 6a.

Reference no. [107] designates the secondary supply inlet connecting the secondary orifice [177] to the secondary supply conduit [106a]. The first secondary and the second secondary supply conduits are referenced as [106a′] and [106b″] respectively. Collectively this fluid conduit network creates an emulsifying device. The actual emulsion is created when an aqueous solution from primary supply conduit [103], is mixed with oil from secondary supply conduit [106a, a′ and b″] at the first fluid junction [120].

Thus, as illustrated in FIGS. 5 and 6, the fluid conduit network of the microfluidic device (or cartridge) comprises a plurality of supply conduits comprising a primary supply conduit [103], a secondary supply conduit [106], a transfer conduit [112], and a first fluid junction [120] providing fluid communication between the primary supply conduit [103], the secondary supply conduit [106], and the transfer conduit [112].

Further, FIG. 4 shows that each group of containers [171] comprises a plurality of containers comprising an intermediate chamber [174], a collection container [134], and one or more supply containers. The one or more supply containers may be separate containers or the primary and the secondary supply containers of a single group of containers may be integrated to form a combined supply container [131] defining one or more supply cavities.

The secondary or the combined supply container [131] comprise a secondary supply orifice [177] extending from the supply cavity [131a].

The collection container [134] being in fluid communication with the transfer conduit [112] of the corresponding emulsification unit [170] via a collection orifice [118] of the collection container.

The secondary supply container is in fluid communication with the secondary supply conduit [FIG. 6, ref. nos. 106a, 106a′, 106b″] of the corresponding emulsification unit [170] via the secondary supply orifice [177].

The secondary or combined supply container [131] is in fluid communication with the intermediate chamber [174] of the same group of containers via the primary orifice [176], and the intermediate chamber [174] is in fluid communication with the first fluid junction [120] of the corresponding emulsification unit [170] via the primary supply conduit [103] of the corresponding emulsification unit [70].

In a preferred embodiment the primary supply conduit [103] have a serpentine-shaped part from the intermediate chamber [174, 174a] to the first fluid junction [120].

Also, the intermediate chamber may have a serpentine-shaped part [FIG. 5b, 174a].

However, both the intermediate chamber [174] and the primary supply conduit [103] may be differently shaped depending on the hydrophobicity and viscosity of the actual reactants used in the emulsification-reaction and the type of material the microfluidic device is made from.

Whereas the preferred device is assembled from 3 parts the microfluidic device may be formed otherwise. As illustrated in FIGS. 7 and 8 a functional microfluidic device (or cartridge) may e.g. be made from only an upper and a lower part.

FIG. 7a illustrate the upper part [382a] and the lower part [382b], seen from the top. FIG. 7b show upper part [380a] and the lower part [380b], seen from the bottom.

The cross-section X-X′ of a group of containers [171] is further explained in FIG. 8a.

Some of the functional different parts of the device is indicated by boxes drawn with a broken line. The supply container [131], the supply container cavity [131a], the collection container [134] and the emulsification unit [170] are indicated.

The supply container [131] defines a supply cavity [131a], it comprises a primary orifice (or inlet site) and a secondary orifice (secondary inlet site) referred to by reference number [176] and [177] in FIG. 8b.

Similarly, to the embodiment in FIG. 4, the primary orifice [176] of the cartridge is configured for forming a seal with a pipette tip when tip is pressed against the primary orifice [176]. Thus, the first primary perimeter [176a] of the primary orifice [176] may gradually become narrower towards the second primary perimeter [176b]. This concept is illustrated in FIG. 8b, FIG. 10b and FIG. 10c.

Both in case of the three- and two-layered embodiment of the microfluidic device, the distance from the first primary perimeter [176a] to the second primary perimeter [176b] may be less than 10 mm such as less than 3 mm.

Similarly to the situation for the three-layered embodiment of the microfluidic device the secondary orifice [177] of the supply container [131] in the two-layered embodiment may extend from a first secondary perimeter [177a] bordering the secondary supply cavity [131a] to at least a second secondary perimeter [177b] to form a cone tapering in a direction away from the supply cavity [131a].

The supply container [131] is in fluid communication with an intermediate chamber [174] of the same group of containers via the primary orifice [176].

Each group of containers also comprise an emulsification unit [170] which is in fluid communication both with the intermediate chamber [174] and with the secondary orifice [177]. Also, the collection container [134] is in fluid communication with the emulsification unit [170] via the collection outlet/collection orifice [118].

In certain preferred embodiments the dimension of the conduits of emulsifying unit [170] comprise fluid conduits being in the microscale, such as conduits having a cross-sectional area smaller than 200 μm², such as less than 30 μm², or even less than 5 μm².

Further details and embodiments of the microfluidic device are described in European Application No. 19154947.6, filed on Jan. 31, 2019, which is herein expressly incorporated by reference in its entirety.

In a preferred embodiment the system comprises a device that create a flow of spatially separated single cells. Such a flow of cells may be created by a cytometric device which typically create the flow of spatially spaced single cells by hydrodynamic- or acoustic-assisted hydrodynamic focusing.

Alternatively, the device that provide the flow of spatially spaced single cells may be one of the microfluidic devices described (Reece et al. (2016) Curr Opin Biotechnol. 2016; 40:90-96; Wen et. al. 2016; 21(7):881.)

The technique of microfluidic InkJet-type single-cell dispenser devices (e.g. Cytena GmbH, Freiburg, Germany) or acoustic- or microvalve bioprinters are other technologies that may be used in the system to provide single cells, at previously defined sites, e.g. inlet sites of the cartridge (Gross, et al. (2015). Int. j. of molecular sciences. 16. 16897-919)

Further examples of focused ejection cell systems that can be adapted to provide volumes comprising a single cell, at the inlet site of the cartridge, comprise a piezo-driven droplet generating devices (EP 2 577 254 B1), optical tweezer devices, acoustic tweezer device (US 2005/0130257 Al), and photoacoustic tweezer devices.

It is contemplated that the part of the system that provides a flow of single localized cells which ejects from the device, one cell at a time and which provides one or more single localized volumes, each comprising a single cell, at a previously defined inlet site (or orifice) of a cartridge (microfluidic device), may be replaced by a single cell picking device that place one or more single cells at a specific inlet site (or orifice) of the cartridge. One example of such single cell picking devices that may be adapted for this purpose is the CytoTrack/CytoPicker system of CytoTrack, Lyngby, Denmark. This system is described in US20180119086A1 and elsewhere.

To obtain amplification of polynucleotides from a single cell, the system is designed to form an emulsion of droplets in which the actual amplification reaction occurs. Accordingly, the cartridge (the microfluidic device) [100], fits into a device which facilitate the formation of an emulsion of droplets by enabling passage of reactants from the supply container [131] through emulsification unit [170] to the collection container [134] of the cartridge [100]. The Xdrop instrument (item #IN00100-SF002, Samplix ApS, Herlev, Denmark) is designed to perform this task.

In further embodiments of the invention, there is provided an assembly comprising the microfluidic device, a thermal structure, and a holder [193] configured to hold the cartridge (microfluidic device) and provide a thermal connection [194] between the thermal structure and a bottom part of the microfluidic device [100]. Such an assembly allows to keep the temperature of the various reactants in microfluidic device at a reduced temperature until the emulsion has formed in the collection well and accordingly reduce possible erroneous amplification reactions to occur before droplet formation.

To increase ease of use and further reduce the risk of contamination, it is contemplated to assemble the entire system into one integrated unit that will be able to perform all the procedures of the separate parts. Such an integrated unit would comprise the device providing a flow of single localized cells, the device that provides one or more single localized volumes, each comprising a single cell, at a previously defined inlet site of a cartridge (microfluidic device) [100], the microfluidic device, and the device facilitating the formation of an emulsion of droplets.

The above described system is specifically designed for amplification of polynucleotides from a predefined number single cell by a method, which comprise the steps of: 1) providing a sample for cells, 2) preparing a microfluidic device or cartridge designed to facilitate formation of an emulsion of droplet by pipetting a volume of cell lysing buffer into or onto an inlet site of the microfluidic device, 3) inserting the cartridge into the device that deposit the predefined number of single cells into the cell lysing buffer at the microfluidic device, 4) applying further reactants and use the microfluidic device to form an emulsion of droplets containing a polynucleotide amplification mix, and 5) incubate the emulsion of droplets to obtain amplified nucleic acid from the predefined number of single cells.

As schematically illustrated in FIG. 9 the method may comprise the individual steps of 1) providing a sample for cells, and 2) treating the sample of cells to obtain a suspension of essentially single cells.

Then, 3) as shown in FIG. 9 panel A and B, a microfluidic device or cartridge is prepared by pipetting a small volume of cell lysing buffer [201] into the cavity of the primary orifice [176].

As illustrated in FIG. 9 panel C the next step, step 4), is by use of the system [01], to eject one or more volumes, each comprising one single cell [06] into the small volume of cell lysing buffer [201].

After a short period of incubation, step 5), during which the cell is lysed and the nucleic acids are released, next step in the method would typically be 6) to pipet a volume of neutralization buffer [202], which is sufficient to neutralize the cell lysing buffer, into the cavity of the primary orifice [176] and the intermediate chamber [174] FIG. 9 panel D, and let the mixture incubate briefly (e.g. 10-60 seconds).

The in step 7), FIG. 9 E, while using a wide-bore pipette-tip [203] configured to form a seal with the distal end zone of the primary orifice [176] when the pipette-tip is inserted correctly into the primary orifice and firmly pressed against the primary orifice, see FIG. 10, a volume of amplification mixture buffer [204], e.g. 15 μl, is pipetted down into the cavity of the primary orifice [176] and the intermediate chamber [174]. By applying sufficient pressure to the pipette the liquids are forced down into the primary orifice [176] and the intermediate chamber [174], FIG. 9F.

Next, in step 8) FIG. 9G, a volume of emulsion oil [205] is added to the combined supply container [131].

The emulsion oil may be any type carrier fluid which is sufficiently immiscible with water to be able to form a water-oil emulsion of aqueous droplets. The carrier fluid can be a non-polar solvent, decane, fluorocarbon oil, silicone oil or any other oil (for example mineral oil). A fluorocarbon oil is preferred.

In certain embodiments, the carrier fluid contains one or more additives such as agents which increase, reduce, or otherwise create non-Newtonian surface tensions (surfactants) and/or stabilize droplets against spontaneous coalescence or contact. Exemplary surfactants that may be used include, but are not limited to, surfactants such as sorbitan-based carboxylic acid esters (e.g., the “Span” surfactants, Fluka Chemika), including sorbitan monolaurate (Span 20), sorbitan monopalmitate (Span 40), sorbitan monostearate (Span 60) and sorbitan monooleate (Span 80). Other non-limiting examples of non-ionic surfactants which may be used include polyoxyethylenated alkylphenols (for example, nonyl-, p-dodecyl-, and dinonylphenols), polyoxyethylenated straight chain alcohols, polyoxyethylenated polyoxypropylene glycols, polyoxyethylenated mercaptans, long chain carboxylic acid esters (for example, glyceryl and polyglycerl esters of natural fatty acids, propylene glycol, sorbitol, polyoxyethylenated sorbitol esters, polyoxyethylene glycol esters, etc.) and alkanolamines (e.g., diethanolamine-fatty acid condensates and isopropanolamine-fatty acid condensates).

When the carrier fluid predominantly consists of fluorocarbon oils, fluorinated surfactants such as Zonyl™ (Dupont, Wilmington Del., USA) or FluoSurfN (prod. #1803-001, Emulseo, Bordeaux Pessac, France) are preferred. In the next step, step 9) FIG. 9H, the cartridge or microfluidic device is inserted into the device [207] facilitating the formation of an emulsion of droplets [206] and the device is activated, step 10).

The device [207] facilitates the formation of the emulsion by supplying pressure (or vucuum) to the microfluidic device (cartridge) [100] whereby the various liquids, in a carefully controlled manner, flow through the emulsification unit of the cartridge and forward to the collection well [134] where the emulsion [206] and the excess oil [205] ends. To obtain the correct pressure conditions during the process the cartridge [100] may be provided with a gasket before inserted into the pressure providing device [207]. A suitable device which facilitate the formation of an emulsion of droplets in the microfluidic device (cartridge) [100] is marketed under the tradename Xdrop (item #IN00100-SF002, Samplix, Herlev, Denmark).

Next step, 11) FIG. 91, is to transfer the oil and emulsion mixture formed in the collection container [134] to a suitable container (e.g. a PCR tube), and, in step 12) FIG. 93, carefully remove most of the excess oil from the container (PCR tube). In a preferred embodiment of the method the carrier fluid is a fluorocarbon oil (e.g. Novec HFE-7500, 3M Co., Maplewood, Minn., USA) resulting in that excess oil may conveniently be removed from the bottom of the collection container (PCR tube).

To accomplish amplification, the emulsion of amplification mix comprising droplets are incubated as prescribed by the applied amplification method. In a preferred embodiment of the method the nucleic acids of the droplets are amplified by method of multiple displacement amplification (MDA). MDA is typically based on the enzyme Phi29 polymerase derived from bacteriophage ϕ29. Main suppliers of Phi29 polymerase such as Qiagen GmbH, Hilden, Germany or Fidelity Systems, Gaithersburg, Md., USA state in their product specifications that the minimum input for the Phi29 polymerase reaction should be 1 ng or higher (www.giagen.com/dk/products/catalog/sample-technologies/dna-sampletechnologies/genomic-dna/repli-g-single-cell-kit), (www.fidelitysystems.com/phi29_hexamers.html). However, surprisingly the Phi29 polymerase was found to provide an effective amplification of DNA molecules using the method of the invention, even when the input DNA was very low, e.g. below 5 fg of DNA. We ascribe this to the very low volumes of the individual droplets formed by the system.

In case MDA is the preferred method of amplification, the next step, step 13) FIG. 9L, would typically be to incubate the emulsion (droplets) at approximately 30° C. for approximately 16 hours followed by ca. 10 minutes at 65° C. in a suitable temperature incubating device [208].

However, the present invention is not confined to situations aimed at amplifying the nucleic acid contents of a single cell solely by the method of multiple displacement amplification.

Subsequent to minor adjustments to the method, the system and the adapted method may be used both for a wide range of isothermal amplification methods of polynucleotide amplification as well as for non-isothermal amplification methods e.g. polymerase chain reaction (PCR).

To obtain the amplified nucleic acid molecules for further analysis, a special break solution [209] may be added to each tube, FIG. 9M, the tubes may be gently mixed, and briefly centrifuged. In case the carrier fluid is a fluorocarbon or similar high-density oil, the lower organic phase can be removed to obtain the upper water phase that will contain the amplified nucleic acid [210] in step 16), FIG. 9N.

In an embodiment, the microfluidic device produces 10,000 to 500,000 droplets per sample such as 30,000 to 100,000 droplets. However, when the number of DNA fragments is high, such as more than 100,000 fragments per sample, it may be preferred to use microfluidic devices that produces more than 30,000 droplets such as more than 500,000 or even more than 2 million droplets per sample. A more even amplification will be obtained if the number of fragments arising from the cell does exceeds the number of droplets. However, a higher number of droplets is expected to yield lower amounts of amplified DNA if the number of fragments arising from the cell is lower than the number of droplets. It may therefore be preferred to produce droplets at a ratio of between 0.1 to 10 DNA fragments per droplet.

It will be appreciated, that the functionality of the invention is critically dependent on the actual microfluidic device and the reactants used, accordingly a kit of parts for carrying out the method is provided.

In one preferred embodiment the kit of parts comprises one or more microfluidic devices (cartridges), each of which comprise one or more groups of containers, wherein each group of containers comprise a supply container, defining a supply cavity and comprising a primary orifice (or inlet site), an emulsification unit and a collection container, each group of containers comprise a plurality fluid conduits that provide for fluid communication between the primary orifice, the emulsification unit and the collection container; a vial of a suitable oil; and a vial of break solution in an amount sufficient to perform the number of reactions provided for by the one or more microfluidic devices (cartridges).

In a further preferred embodiment the kit of parts further comprises a holder and one or more gaskets for the one or more microfluidic devices (cartridges) to fit into the device [207] facilitating the formation of an emulsion of droplets [206]. The kit may further comprise a vial of amplification mix and a vial of enzyme in an amount sufficient to perform the number of reactions provided for by the one or more microfluidic devices (cartridges).

The following represents a list the references of the drawings.

Any relevant part of the above disclosure may be understood in view of the below list of references in combination with the disclosed drawings.
[01] The system
[02] device (or part) ejecting single cells
[03] sample handler
[04] Flow of single localized cells
[05] focused ejection cell system device
[06] one single cell
[06] single cells
[07] Sample of cells
[08] Light source/laser
[09] the detector
[10] Sheath part of hydrodynamic focusing device
[100] cartridge or microfluidic device
[103] primary supply conduit
[104] primary supply inlet
[106] secondary supply conduits
[106a] secondary supply conduits
[106a′] first secondary supply conduit
[106b″] second secondary supply conduit
[107] secondary supply inlet
[112] transfer conduit
[118] collection orifice
[120] fluid junction
[120] first fluid junction
[131] supply container (combined)
[131a] supply container cavity
[132] primary supply container
[133] secondary supply container
[134] collection container or well
[135] fluid conduit network
[135a] broken line box indicate the location of the emulsifying microfluidic network at the lower part of the device
[135b] broken line box indicate the location of the emulsifying microfluidic network at the middle part of the device
[170] emulsification unit
[171] group of containers
[174] intermediate chamber
[174a] serpentine-shaped part
[176] primary orifice (or inlet site)
[176a] primary perimeter
[176b] second primary perimeter
[177] secondary orifice (secondary inlet site)
[177a] first secondary perimeter
[177b] second secondary perimeter
[180] lower piece of microfluidic device
[180a] lower piece of microfluidic device seen from the top
[181] intermediate piece of microfluidic device
[181b] middle part of microfluidic device
[182] upper piece of microfluidic device
[182a] upper piece of microfluidic device seen from the top
[182b] upper piece of microfluidic device seen from the bottom
[190] handle
[193] holder
[194] thermal connection
[201] lysing buffer
[202] neutralization buffer
[203] wide-bore pipette-tip
[204] amplification mixture buffer
[205] emulsion oil
[206] emulsion of droplets
[207] device which facilitate the formation of an emulsion of droplets
[208] temperature incubating device
[209] break solution
[210] amplified nucleic acid
[380a] upper part of microfluidic device seen from the bottom
[380b] lower part of microfluidic device seen from the bottom
[382a] upper part of microfluidic device seen from the top
[382b] lower part of microfluidic device seen from the top
[400] FACS adaptor with a dMDA cartridge inserted
[401] FACS adaptor
[402] Top plate of FACS adaptor
[403] Middle plate of FACS adaptor
[404] Bottom plate of FACS adaptor
[405] Vertical sliding plate of FACS adaptor
[406] FACS attachment element
[407] protrusions at bottom of the top plate

### EXAMPLES

Example 1: Multiple displacement amplification (MDA) performed in droplets show considerably less loss of information than a standard MDA reaction.

In this example MDA performed in droplets is compared with the performance of two standard bulk MDA reaction kits.

Materials and Methods.

DNA-template.

Purified Escherichia coli chromosomal DNA (Affymetrix (Thermo Fisher Scientific), Santa Clara, Calif., USA)

Bulk_MDA Kits:

Q: REPLI-g Mini Kit, Cat No./ID: 150025; (Qiagen GmbH, Hilden, Germany).

N: phi29 DNA Polymerase (M0269) (New England Biolabs, Ipswich, Mass., USA, NEB)/using NEB phi29 DNA Polymerase Reaction Buffer NEB Catalog #B0269.

Droplet MDA Kit:

Samplix′ dMDA Kit (item #RE20300)

Cartridge/Microfluidic Device:

Samplix, Herlev, Denmark (item #CA20100) including dMDA holder (Samplix item #HO10100) and dMDA gasket (Samplix item #GA20100).

Bulk_MDA Protocols:

According to the instructions of the manufacturers.

Droplet MDA Protocol (dMDA) in Brief:

1) Mix DNA and reagents, 2) Load sample and oil onto cartridge and insert cartridge into the Xdrop™ instrument (Samplix item #IN00100-SF002)—40 seconds droplet generation, 3) Incubation of droplets at 30° C. for 8-16 hours, 4) Break droplets and transfer amplified DNA to a new tube for library preparation. However, the instructions of the manufacturer were closely followed.

Library-Construction and Sequencing:

Was performed by Eurofins Genomics, Ebersberg, Germany

Relative Coverage:

The relative coverage was calculated and used to benchmark target coverage in dMDA vs bulk MDA.

In brief, after alignment the relative coverage was calculated by counting the number of aligned nucleotides of the sequenced reads when compared to the corresponding section in the reference E. coli genome.

Specifically, after alignment of the sequenced reads to the reference genome the total number of aligned nucleotides (Nₜₒₜₐₗ) for all positions (p) in the reference genome of length l was calculated:

$${N}_{total} = {\sum }_{p = 1}^{l} {N}_{p}$$

Then the average coverage across the entire reference sequence was calculated by dividing the total number of aligned nucleotides (Nₜₒₜₐₗ) with the length of the reference genome (I):

N_{avg}=Nₜₒₜₐₗ/l

The relative coverage (cov) at the center of each bin was calculated as the sum of aligned nucleotides within the positional window (from start to start+width) divided by the average coverage (N_{avg}) corrected for the window width (width):

$$cov = \frac{{\sum }_{p = start}^{start + width} {N}_{p}}{{N}_{avg} \cdot width}$$

And from this the log base10 relative coverage was calculated (if it is defined, i.e. if there are any alignments in the window):

$${cov}_{{log}_{10}} = \{ \begin{matrix}{log}_{10} ( cov ) , & if cov > 0 \\ NaN & otherwise\end{matrix}$$

Results

E. coli DNA was amplified using dMDA (Samplix) and bulk amplification products from two commercial suppliers, Q and N, and the amplified DNA was sequenced using Illumina sequencing, Illumina, Inc., San Diego, Calif., U.S.A.

The relative coverage of the E. coli sequence obtained by 3 methods was calculated and plotted. FIG. 11-14 show the results when various amounts of chromosomal DNA were subjected to amplification by one of the three protocols. Log base10 relative coverage for an unbiased amplification is scored to 0 (relative coverage is 1). When the amount of chromosomal DNA was 1 pg, only the droplet MDA protocol provided a specific signal. This is further emphasized from the results shown in FIG. 15. It should be noted that even at the very low amount of 1 pg DNA, the droplet MDA amplification was significantly less biased compared to the results obtained with the bulk MDA protocols. Close to 100% of the amplified DNA mapped to E. coli, indicating a very high specificity (FIG. 15a). It is also noteworthy the dMDA protocol amplify DNA in a clear dose dependent fashion (FIG. 15a), suggesting that dMDA may be used for quantitative studies.

Conclusion

This example demonstrated that MDA performed in droplets provided a significantly more sensitive and unbiased amplification when compared with two standard MDA bulk reaction schemes.

Example 2: An adaptor conveying a Samplix dMDA cartridge at a FACS, a Sony Cell Sorter SH800S.

This example illustrate a manually adjustable adaptor that fit a dMDA cartridge (Samplix ApS, Herlev, Denmark, Cat. #CA20100) inside the sample collection area of a Sony Cell Sorter SH800S FACS.

The FACS adaptor was manufactured in PA2200 (polyamide 12) plastics by 3D printing. The actual 3D printing was performed by Damvig A/S, Taastrup, Denmark.

FIGS. 21 and 22 show the FACS adaptor with an a dMDA cartridge (Samplix ApS, Herlev, Denmark, Cat. #CA20100) inserted. The middle plate [403], the bottom plate of FACS adaptor [404], and the vertical sliding plate [405] of the FACS adaptor are interlocked to allow for adjustments somewhat similar to the mechanisms seen in cross slide- or X-Y-tables.

The protrusions [407] extending from the bottom of the top plate of the FACS adaptor [402] fits into an array of holes in the middle plate [403] facilitating the convenient positioning of the dMDA cartridge in 8 different positions aligning any of the 8 primary orifices (or inlet sites) [176] of a dMDA cartridge with the stream of cells ejected from the FACS.

Example 3: Amplification of DNA from a single cell by use of the system and method using a cartridge that creates single emulsion droplets.

This example describes how single cells were deposited into the microfluidic device using a FACS adaptor (see example 4) for the Samplix dMDA cartridge. The cells were lysed, their content of polynucleotides denatured and then neutralized. The cells are mixed with dMDA reagents, followed by droplet formation, dMDA incubation and isolation of genetic material. The amplified DNA was randomly fragmented by sonication and used as a substrate in DNA library generation for both Whole-Exome Sequencing (WES) and Whole-Genome Sequencing (WGS). DNA Libraries from the amplified cell material were sequenced by an Illumina Novaseq 6000 and the data analyzed with relevant software. The results of sequencing of DNA from single cells amplified with droplet-based MDA and using Samplix (Herlev, Denmark) reagents, DNA from single cells amplified by conventional MDA using Samplix reagents but without droplet formation, or by conventional MDA using a commercially available MDA kit without droplet formation were compared. Further the sequences obtained from amplified single cells amplified were compared to unamplified DNA isolated from a large batch of cells from the cell line in question. Analysis include evaluation and comparison of GC-percentages, mapping to the reference genome, and the sequence coverage across the genome.

Materials and Methods

The Droplet MDA Method (dMDA)

1. Adjusting Samplix dMDA Cartridge FACS Adaptor for Single Cell Sorting.
Before sorting, the cell sorter instrument (FACS, a Sony Cell Sorter SH800S) and the FACS adaptor was subjected to a series of presorting tests to adjust the FACS adaptor.
The first test was a spray test to validate that sorting was precise and sorted droplets only formed one droplet.
Next, the FACS adaptor equipped with a dMDA cartridge was placed in the FACS instrument. The cartridge inlets were covered with foil and the position of the primary orifices of the dMDA cartridge were marked on the foil. A predefined number of beads (eg. 100 pieces, Alignflow™ Flow Cytometry Alignment Beads for Blue Lasers, 6.0 μm) were then sorted onto the foil and the FACS adaptor was adjusted. The adjustment was repeated until the sorted material hit exactly on the mark of each primary orifice.

2. Single Cell Sorting Validation Assay
To validate the adjustment of the system a mixture of beads (4000 beads/ml) and Horseradish Peroxidase (HRP; 250 ng/μl) was used to simulate the sample and was sorted by the FACS using Single Cell mode. The beads were singly deposited directly into 10 μl TMB (3,3″,5,5″-tetramethylbenzidine) at the dMDA cartridges primary orifice/inlet site.
If the beads were sorted correctly, the inlet turned blue after 30 minutes of incubation at room temperature in darkness.

3. Cell Sorting and Lysis.
A single cell (HT29 human colon cancer cell) was deposited directly into 2.8 μL lysis buffer (200 mM KOH, 0.5 M EDTA and 40 mM DTT) at each of the inlet sites/primary orifices [176] of the dMDA cartridge by use of the FACS. The single cells were lysed, and DNA denatured for 5 minutes at room temperature. Then 1.4 μL neutralization buffer (400 mM HCl and 600 mM Tris HCl (pH 7.5)) was added and incubated for 5 min at room temperature.
Using a wide bore pipette-tip with filter (e.g. Rainin BioClean Ultra Wide-O, Cat. #30389241), 15.8 μL MDA amplification mixture including polymerase primers, dNTP and reaction buffer (Samplix dMDA kit Cat. #RE20300), was added to the sample, by injecting it into the inlet site. Then 75 μL dMDA oil (Samplix dMDA kit Cat #RE20300) was added into the inlet well (supply cavity).

4. MDA Droplet Generation.
The dMDA cartridge was then moved into the Xdrop™ droplet generator (Samplix ApS, Herlev, Denmark, Cat #IN00100-EU/US) and single emulsion droplets were formed according to the manufacturer's instructions. Droplets were removed from from the collection container/well of the dMDA cartridge, transferred to low bind vials (e.g. Eppendorf DNA LoBind tubes, Cat. #022431005) and excess oil was removed.

5. MDA in Droplets:
The MDA droplets were then incubated in a thermal block at 30 degrees Celsius for 16 hours and the reaction was stopped by incubating at 65 degrees Celsius for 10 minutes.

6. Breaking of Droplets:
Subsequently droplets were broken (coalesced) by adding 20 μL break solution and 1 μl break colour (Samplix dMDA kit Cat #RE20300, Samplix ApS, Herlev, Denmark). The sample tubes were flicked a few times, spun at 5000 rpm and the oil was removed from the sample.

7. DNA QC:
DNA material was quantified in-house using a Quantus™ Fluorometer (Promega Corp, Madison, Wis., USA) and the integrity investigated using Agilent 4200 TapeStation (Agilent Inc., Santa Clara, Calif., USA) according to the manufacturer's instructions.

Bulk MDA

Bulk_MDA amplification was performed using the following protocol:
1. Deposit 2.8 μl lysis buffer (200 mM KOH, 0.5 M EDTA and 40 mM DTT) in each tube (Keep lids closed and tubes cold to avoid evaporation).
2. Sort 1 HT29 cell into the tube.
3. Spin the tube after sort.
4. Incubate for 5 min at RT.
5. Add 1.4 μl Neutralization buffer (400 mM HCl and 600 mM Tris HCl (pH 7.5)) to tube.
6. Incubate for 5 min at RT.
7. Place the tubes in a cooling block until the MDA mix has been made.
8. Add 15.8 μl MDA amplification mixture including polymerase, primers, dNTP and reaction buffer (Samplix dMDA kit Cat. #RE20300) and mix gently by pipetting.
9. Incubate the tubes in a thermo cycler as described in the table below.

| Temperature | Duration |
|---|---|
| 30° C. | 16 hours |
| 65° C. | 10 minutes |
| 4° C. | Until stop |

REPLI-g Batch Amplification
The REPLI-g batch amplification of DNA from a single cell was performed by use of the Qiagen REPLI-g Single Cell Kit (Cat No./ID: 150343) while closely following the manufacturer's (QIAGEN GmbH, Hilden, Germany) manual and recommendations.

Bulk-MDA Batch Amplification
The Bulk-MDA batch amplification of DNA from a single cell was performed by use of the by use of the Samplix′ dMDA Kit (Cat. #RE20300) and following the manufacturer's (Samplix, Herlev, Denmark) recommendations but without prior droplet formation.

Sequencing

Next Generation Sequencing.
DNA libraries were generated for both Whole Exon Sequencing (WES) and Whole Genome Sequencing (WGS) using DNA library kits (Agilent SureSelect Human All Exon V6 and 350 bp insert DNA library, respectively) and the samples run on a Novaseq 6000 (Illumina Inc., San Diego, Calif., USA). The sequencing data output was approximately 6 Gbp raw data per sample for the WES and 90 Gbp raw data per sample for the WGS.
Approximately 200 million reads were generated from each sample. The actual sequencing was performed by Novogene Co., Ltd, UK. Sequence data was then collected and analyzed using standard methods.

GC %
The GC % was determined as a part of the service provided by Novogene Co., Ltd, UK.

Results

DNA from a single HT29 human colon cancer cell line cells (established from a primary adenocarcinoma in a 44 year old Caucasian female) was amplified using the method of the invention, the single cell Xdrop™ dMDA technology or “dMDA”.

The GC % observed in DNA amplified by the present invention (Xdrop method) was compared to the GC %'s obtained on DNA amplified by: 1) a golden standard for MDA amplification of DNA from only a single cell (Qiagen REPLI-g Single Cell Kit); 2) DNA from a single cell amplified using essentially the same enzymes and the same protocol as used in the Xdrop™ dMDA-protocol but omitting droplet-formation; and 3) and the GC % of an unamplified DNA sample. See FIG. 16.

Except from the single unamplified DNA sample, all datapoints represent 4 individual samples.

The data show that the Whole-Exome Sequencing (WES) GC % of DNA from single cells and amplified by the method of the present invention is similar to the WES GC % of a sample of non-amplified DNA. Interesting, also the WES GC % of DNA from the two “batch”—methods, the “Bulk_MDA” and the “Golden_Std_MDA” are rather similar, but different from the unamplified DNA.

According to literature the GC % of the human genome is is about 41% (Piovesan, A. et al. (2019) BMC research notes vol. 12, 106) whereas the GC content in exons is about 50% (Meienberg, J. et al. (2016) Human genetics, 135(3), 359-362; Amit, M. Et al (2012) Cell Reports 1, 543-556).

This suggests that the present droplet-based method provide a less biased amplification compared both to a market standard method of single cell DNA amplification and even when compared to a bulk DNA amplification method using same enzymes and—except for droplet-formation—same protocol as the method of the invention.

FIG. 17 shows the mean coverage depth per chromosome across the human genome as it was calculated from the sequencing data obtained by Whole Genome Sequencing (WGS) performed by Novogene Co., Ltd., UK. WGS sequence data was obtained from a) unamplified genomic DNA; two batch-amplified DNA samples—amplified either b) by use of the Samplix′ dMDA Kit (Cat. #RE20300) or c) by the Qiagen REPLI-g kit; and d) by the present invention were compared.

The unamplified genomic DNA (FIG. 17A) shows the least biased coverage across the chromosomes, followed by amplification performed on an emulsion of droplets (the Xdrop dMDA samples, FIG. 17d). DNA amplified without prior droplet formation, FIGS. 17b and 17c, seem to be more variable and thus more biased in terms of coverage across the genome compared to the present invention, the Xdrop dMDA protocol.

This is further illustrated in FIGS. 18a and 18b.

Also the actual coverage varied between the methods. The close-up of FIG. 18b shown in FIG. 19 indicates this point. Some of the REPLI-g amplifications (broken line) appear not to cover chromosome 1, 2, 3, 4, 6, 8, 11, 12, 13, 15, 17, 19, 20 and 21, whereas only one X-drop sample missed data from one chromosome, chromosome 22. As expected no chromosome Y related DNA sequences ware seen in any of the experiments (HT29 is from a female). Thus, the present invention provides an amplification which is less biased, and which enables more even coverage of the exons.

The results shown in FIGS. 20a and 20b further show that one obtains a higher sequence depth with the present invention. FIG. 20a shows that approximately 50% of the unamplified DNA have an sequence depth of 4.5 or less, whereas approximately 50% of the exome DNA nucleotides resulting from amplification by the droplet-based MDA protocol according to the present invention have a sequence depth of 8 and higher. As shown in FIG. 20b the larger sequence depth provided for by the present invention seems to be caused by the droplet formation included in the protocol.

Thus, the present invention provisde an amplification which is less biased, which enables a more even and complet coverage, and which provide a higher sequence depth than a protocol that do not comprise droplet formation.

Example 4: Amplification of DNA from a single cell by use of the system and method using a cartridge that creates Double emulsion droplets.

By applying a double emulsion-creating cartridge, e.g. the Samplix cartridge described in PCT/EP2020/052400 (Cat. No. CA10100) and a modified FACS adaptor the system may easily be adapted to comprise a cartridge that create double emulsion droplets.

Materials and Methods

Much of the materials and methods will be similar to those applied when using a single emulsion cartridge, see example 3.

Similarly to what is described in example 3, the double-emulsion cartridge will be inserted into a FACS adaptor, placed in a FACS and adjusted to ensure that a single cell is deposited into a small volume of lysis buffer at each inlet orifices of the primary supply container of the cartridge.

The cartridge will be removed from the FACS and the single cells allowed to lyse. Then the lysis buffer will be neutralised, and the MDA amplification mixture including polymerase primers, dNTP and reaction buffer (e.g. Samplix dMDA kit Cat. #RE20300) added to the samples. The remaining reactants, e.g. dMDA-buffer and dMDA oil are added (in case the double emulsion-creating cartridge used is the Samplix Cat. No. CA10100, the remaining reactants may be added to the cartridge before inserted into the FACS). The fully loaded cartridge is then inserted into a droplet-forming instrument, e.g. the Xdrop™ droplet generator (Samplix ApS, Herlev, Denmark, Cat #IN00100-EU/US) and double emulsion droplets formed.

Droplets are then removed from the collection container/well of the dMDA cartridge, transferred to low bind vials (e.g. Eppendorf DNA LoBind tubes, Cat. #022431005) and incubated e.g. in a thermal block at 30 degrees Celsius for 16 hours and subsequently at 65 degrees Celsius for 10 minutes.

Subsequently droplets will be broken by adding break solution, the DNA is recovered, further processed and sequenced.

Expected Results:

While the system using a cartridge that create single emulsion droplets typically results in the creation of some 20.000 droplets, a system that uses a cartridge that creates double emulsion droplets typically result in the formation of some 4-5.000.000 droplets.

The much larger number of droplets obtained in a double emulsion system is expected to show more homogeneous amplification if the number of fragments arising from the cell exceeds the number of droplets. However, a higher number of droplets is expected to yield lower amounts of amplified DNA if the number of fragments arising from the cell is lower than the number of droplets.

1. A system for amplification of polynucleotides from a predefined number of single cells comprising a device (or part) providing a predefined number of single cells, at an inlet site (or orifice) of a cartridge (or microfluidic device), and the cartridge,
said device provides a flow of single localized cells which ejects from the device, one cell at a time, and deposits them at a previously defined inlet site (or orifice) of the cartridge (or microfluidic device),
said cartridge comprises one or more groups of containers,
wherein each group of containers comprise a supply container, defining a supply cavity and comprising a primary orifice (or inlet site),
an emulsification unit and a collection container,
each group of containers comprise a plurality of fluid conduits that provide for fluid communication between the primary orifice, the emulsification unit and the collection container, and between a secondary orifice, the emulsification unit and the collection container.

2. The system according to claim 1, wherein the device, providing single localized volumes, provides volumes, each of which comprising a single cell, at an inlet site of the cartridge.

3. The system according to, claim 1 wherein the device, providing single localized volumes, comprises a focused ejection cell system.

4. The system according to claim 1, wherein the device providing a flow of single localized cells is selected from the group of devices consisting of a flow cytometer, a fluorescence-activated cell sorting device (FACS), a single cell “inkjet” device, an acoustic bioprinter, a piezodriven droplet generating device, and a single cell dispenser.

5. The system according to claim 1, wherein the primary orifice of the cartridge is configured to form a seal with a pipette tip when the distal end of the pipette tip is pressed against the primary orifice.

6. The system according to claim 1, wherein each emulsification unit of the cartridge comprises a fluid conduit network comprising:
a plurality of supply conduits comprising a primary supply conduit and a secondary supply conduit;
a transfer conduit;
and a first fluid junction providing fluid communication between the primary supply conduit, the secondary supply conduit, and the transfer conduit;
each group of containers comprises a plurality of containers comprising an intermediate chamber, a collection container, and one or more supply containers,
wherein the supply container comprises a supply container cavity and wherein the supply container comprise a primary orifice extending from the supply cavity,
the secondary supply container is optionally a separate container or integrated with the primary supply container forming a combined supply container, defining one or more supply cavities,
the secondary or the combined primary and secondary supply container 20 comprise a secondary orifice extending from the supply cavity, the collection container being in fluid communication with the transfer conduit of the corresponding emulsification unit via a collection orifice of the collection container,
the supply container is in fluid communication with the secondary supply conduit of the corresponding emulsification unit via the secondary orifice,
the supply container is in fluid communication with the intermediate chamber of the same group of containers via the primary orifice, the intermediate chamber is in fluid communication with the first fluid junction of the corresponding emulsification unit via the primary supply conduit of the corresponding emulsification unit.

7. The system according claim 3, wherein the intermediate chamber has a serpentine-shaped part.

8. The system according to claim 3, wherein the primary supply conduit has a serpentine-shaped part between the intermediate chamber and the first fluid junction.

9. The system according to claim 1, wherein the focused ejection cell system comprises a device, selected from the group of devices, consisting of a hydrodynamic focusing device, a piezo-driven droplet generating device, an optical tweezer device, an acoustic tweezer device and a photoacoustic tweezer device.

10. The system according to claim 1, wherein the system forms one integrated unit.

11. The system according to claim 1, wherein the cartridge (or microfluidic device), fits into a device which facilitate the formation of an emulsion of droplets by enabling passage of reactants from the supply container through the emulsification unit to the collection container of the cartridge.

12. The system according to claim 1, wherein the system comprises an assembly comprising the microfluidic device, a thermal structure, and a holder configured to provide a thermal connection between the thermal structure and a bottom part of the microfluidic device, wherein at least a majority of the intermediate chamber of each group of containers may be provided within 5 mm from the thermal structure.

13. A method for amplification of polynucleotides from a predefined number of cells comprising using the system according to claim 1, which comprise the steps of:
i. providing a sample for cells,
ii. preparing a microfluidic device or cartridge by pipetting a volume of cell lysing buffer into or onto an inlet site of the microfluidic device,
iii. inserting the cartridge into the system and deposit a predefined number of single cells into the cell lysing buffer at the microfluidic device,
iv. applying further reactants and use the cartridge to form an emulsion of droplets containing a polynucleotide amplification mix, and
v. incubating the emulsion of droplets to obtain amplified nucleic acid from the predefined number of single cells.

14. The method according to claim 13, comprising the steps of:
1. providing a sample for cells,
2. treating the sample of cells to obtain a suspension of essentially single cells,
3. preparing a microfluidic device or cartridge by pipetting a small volume of cell lysing buffer in the cavity of the primary orifice,
4. by use of the system, ejecting one or more volumes, each comprising one single cell into the small volume of cell lysing buffer, the number of volumes correspond to the predefined number of cells,
5. incubating for a time sufficient to obtain cell-lysis,
6. pipetting a volume of neutralization buffer into the cavity of the primary orifice and the intermediate chamber and briefly incubate,
7. pipetting a volume of amplification mixture buffer down into the cavity of the primary orifice and the intermediate chamber by using a wide-bore pipette-tip configured for forming a seal with the distal end zone of the primary orifice when the pipette-tip is pressed against the primary orifice, while applying sufficient pressure the liquids are forced well into the primary orifice and the intermediate chamber,
8. adding a volume of emulsion oil to the combined supply container,
9. providing the cartridge with a gasket (if necessary) and insert it into the device facilitating the formation of an emulsion of droplets by supplying pressure (or vacuum) to the microfluidic device (cartridge),
10. activating the device to form an emulsion of droplets assembling in the collection container of the cartridge,
11. transferring the oil and emulsion mixture formed in the collection container to a suitable container (e.g. PCR tube),
12. removing the excess oil from the bottom of the collection container (PCR tube),
13. incubating the emulsion (droplets) at the prescribed temperature in a suitable device.
14. adding break solution to each tube and gently mix,
15. spinning tube briefly, and removing the lower organic phase, repeat step if necessary,
16. keeping the upper water phase that will contain the amplified nucleic acid

15. A kit of parts for carrying out a method according to claim 13, which comprises:
a) one or more microfluidic devices (cartridges), each of which comprise one or more groups of containers,
wherein each group of containers comprise a supply container, defining a supply cavity and comprising a primary orifice (or inlet site), an emulsification unit and a collection container,
and wherein each group of containers further comprise a plurality fluid conduits that provide for fluid communication between the primary orifice, the emulsification unit and the collection container; and
b) a vial of a suitable oil and a vial of break solution in an amount sufficient to perform the number of reactions provided for by the one or more microfluidic devices (cartridges).
